(19) United States
(12) Patent Application Publication
(10) Pub. No.: US 2025/0170559 A1
(43) Pub. Date: May 29, 2025

(54) METHOD OF MAKING SUPERABSORBENT POLYMER MATERIAL USING SOLUBLE POLYACRYLIC ACID POLYMERS HAVING DOUBLE BONDS

(71) Applicant: The Procter & Gamble Company, Cincinnati, OH (US)

(72) Inventors: Arsen Arsenov SIMONYAN, Koenigstein im Taunus (DE); Natasa DIJAKOV, Hanau (DE); Dimitris Ioannis COLLIAS, Mason, OH (US); Martin Ian JAMES, Hamilton, OH (US); Yiping SUN, Mason, OH (US); Jonathan Richard STONEHOUSE, Windlesham (GB); Jacqueline Besinaiz THOMAS, Oregonia, OH (US); Gary Wayne GILBERTSON, Liberty Township, OH (US)

(21) Appl. No.: 19/026,921

(22) Filed: Jan. 17, 2025

Related U.S. Application Data
(62) Division of application No. 17/498,908, filed on Oct. 12, 2021, now abandoned.
(60) Provisional application No. 63/092,543, filed on Oct. 16, 2020.

(51) Int. Cl. B01J 20/26; A61L 15/24; A61L 15/60; B01J 20/28; B01J 20/30

(52) U.S. Cl. CPC B01J 20/267; A61L 15/24; A61L 15/60; B01J 20/261; B01J 20/28016; B01J 20/3021; B01J 20/3085

(57) ABSTRACT

A method of making superabsorbent polymer material and comprising the step of providing soluble polyacrylic acid polymers. The soluble polyacrylic acid polymers have a molar percent of carbon-to-carbon double bonds of at least 0.03. The soluble polyacrylic acid polymers may be obtained from pre-existing recycled post-consumer superabsorbent polymer material, and/or from pre-existing recycled post-industrial superabsorbent polymer material. Superabsorbent polymer material obtained by the method, and absorbent articles comprising these materials are also provided.

### CROSS-REFERENCE TO RELATED APPLICATIONS

This application is a divisional of, and claims priority under 35 U.S.C. § 120 to, U.S. patent application Ser. No. 17/498,908, filed on Oct. 12, 2021, which claims the benefit, under 35 USC 119 (e), to U.S. Provisional Patent Application No. 63/092,543, filed on Oct. 16, 2020; each of which are herein incorporated by reference in their entirety.

### FIELD OF THE INVENTION

The present invention is directed to a method for making superabsorbent polymer material wherein the method uses soluble polyacrylic acid polymers have a molar percent of carbon-to-carbon double bonds of at least 0.03. The soluble polyacrylic acid polymers may be obtained from recycled superabsorbent polymer particles which have been (partially) degraded.

Superabsorbent polymer material obtainable by the method are also provided.

### BACKGROUND OF THE INVENTION

The use of superabsorbent polymer material (hereinafter referred to as ‘SAP material’), typically in particulate form (hereinafter referred to as ‘SAP particles’), especially in disposable absorbent articles, is well known in the art. In light of the large quantities of used and disposed absorbent articles, there is a need to find ways to recycle the materials comprised by the absorbent articles. SAP material forms a meaningful portion of the materials comprised by absorbent articles. Hence, recycling of SAP materials from used and disposed absorbent articles is substantial for absorbent article recycling. SAP material derived from used absorbent articles cannot usually be recycled as is but needs to be degraded for recycling. Recently, various methods for SAP material degradation have been developed, including chemical degradation, degradation via UV radiation, ultrasonication, microwave radiation, or mechanochemical degradation.

However, there is a need to recycle and re-use the materials derived from SAP material degradation.

### SUMMARY OF THE INVENTION

SAP materials, such as SAP particles used in absorbent articles are most often made of cross-linked polyacrylic acid polymers. Degradation of cross-linked polyacrylic acid polymers into acrylic acid monomers is generally very energy-and/or time-consuming. Depending on the SAP material degradation method, and also on how much time and/or energy is afforded in a SAP material degradation method, the methods do not necessarily lead to complete degradation, i.e. they do not result in acrylic acid monomers. Instead, the methods facilitate degradation into soluble polyacrylic acid polymers. Hence, the cross-links of the insoluble superabsorbent polymer material are broken up, leading to polyacrylic acid polymers (hereinafter also referred to as “s-PAA polymers”) which are soluble in aqueous solution.

It is known to use polyacrylic acid oligomers in SAP material making, e.g. in combination with acrylic acid monomers. These oligomers will typically polymerize into the crosslinked acrylic acid network of the SAP material. In contrast thereto, it is believed that most polymers of acrylic acid, i.e. molecules with considerably higher molecular weight versus oligomers, do not readily or only to a small extent polymerize into the SAP crosslinked acrylic acid network.

For absorbent articles comprising SAP particles which exhibit good absorbing and containing functions, specific technical requirements need to be fulfilled by the SAP particles, such as sufficient capacity, permeability of the SAP particles. Generally, high capacity and high permeability is desirable. Another important parameter is the amount of extractables of the SAP material. High amounts of extractables are generally not desired for SAP particles, as they negatively impact the performance of the SAP particles. Extractables tend to leach out of the cross-linked polymer network once the superabsorbent polymer material is swollen, thus affecting superabsorbent properties both by loss of superabsorbent mass, and by the osmotic competition of extractables against the insoluble polymer matrix.

It has been found that upon introducing certain s-PAA polymers into SAP particles, the amount of extractables undesirably increases. However, the inventors have further found that s-PAA polymers can be obtained that comprise carbon-to-carbon double bonds. These double bonds have been identified at the ends of the respective s-PAA polymer chains. The s-PAA polymers were obtained by degradation of pre-existing SAP material.

The carbon-to-carbon double bonds were identified using an NMR Alkylene Content Method. The method enables determination of the molar fraction of unsaturated alkylene moieties as a fraction of moles of PAA polymer backbone tertiary proton moieties in a specimen. The method also allows to conclude if the carbon-to-carbon double bonds are present at the end of a polymer chain or rather at some position in the chain that is spaced from the chain end.

By providing s-PAA polymers having such carbon-to-carbon double bonds, the s-PAA polymers are able to react with the other components provided in the method of making the SAP material of the present invention. They can react with the monomers and/or oligomers and thereby are covalently bound into the superabsorbent polymer network. Those s-PAA polymers having carbon-to-carbon double bonds at two or more of their chain ends (notably, branched s-PAA polymers have more than two chain ends) can basically function as crosslinkers within the polymer network. It has even been found that the use of s-PAA polymers having carbon-to-carbon double bonds facilitate the reduction of the traditional cross-linkers in the method of SAP material making, even eliminating the use of cross-linkers.

As the s-PAA polymers are covalently built into the SAP material network, they are not able to leak out of the SAP material upon swelling of the SAP material. Therefore, the amount of extractables can be reduced. At the same time, parameters reflecting capacity (measured as Centrifuge Retention Capacity, CRC) and permeability (measured as Urine Permeability Measurement, UPM) are not adversely affected compared to methods not applying soluble PAA polymers. This has been proven even for SAP material comprising relatively high amounts of soluble PAA polymers.

Different methods for degradation of pre-existing SAP material have been provided previously, such as chemical degradation (e.g. oxidative degradation), degradation using UV and mechanical degradation. Non-limiting examples of SAP degradation methods are processing: in an extensional flow device (e.g. U.S. Patent Application No. 62/890,631); using hydrothermal microwave (e.g. U.S. Patent Application No. 62/890,632); using UV irradiation in a flow system (e.g. U.S. patent application Ser. No. 16/548,873); using sonication/ultrasonics (e.g. U.S. Patent Application No. 62/890,880), using oxidative degradation (European patent application EP2019193221); using supercritical water; using a combination of an extensional flow device, oxidative degradation, and enzymatic degradation (e.g. U.S. Patent Application No. 63/039,496); using an extensional flow device and oxidative degradation (e.g. U.S. Patent Application No. 63/039,498); and any combination thereof.

The inventors have identified that whether or not soluble polyacrylic acid polymers having carbon-to-carbon double bonds are obtained by chemical degradation depends on the degradation method. Also, the extent of carbon-to-carbon double bonds (i.e. the molar percent of carbon-to-carbon double bonds) depends on the degradation method. Chemical degradation, especially oxidative degradation, has been found to work especially well, such that the soluble polyacrylic acid polymers obtained by such degradation yields a high molar percent of carbon-to-carbon double bonds.

No carbon-to-carbon double bonds have been identified in commercially available soluble polyacrylic acid polymers.

The invention relates to a method of making superabsorbent polymer material. The method comprises the steps of
a) providing an aqueous solution of polymerizable acrylic acid monomers and/or polymerizable acrylic acid oligomers, optionally neutralizing at least some of the polymerizable acrylic acid monomers and/or polymerizable acrylic acid oligomers;
b) optionally providing one or more ethylenically unsatured co-monomers, optionally neutralizing at least some of the ethylenically unsatured co-monomer of step b);
c) optionally providing one or more crosslinker(s);
d) providing one or more initiator(s);
e) providing soluble polyacrylic acid polymers, wherein the soluble polyacrylic acid polymers have a molar percent of carbon-to-carbon double bonds of at least 0.02, preferably at least 0.04, more preferably at least 0.05, still more preferably at least 0.08, and even more preferably at least 0.1;
f) mixing the aqueous solution of monomers, oligomers, co-monomers, crosslinkers and initiators and soluble polyacrylic acid polymers provided in steps a) to e); and
g) polymerizing the mixture obtained in step f) to obtain a superabsorbent polymer.

The superabsorbent polymer material obtained by the method may have a ratio of the difference between extractables [weight-%] and add-on level of s-PAA polymer [weight-%] to base polymer capacity (in g/g, measured as CRC according to the test method set out herein) of less than 0.15, or less than 0.12, or less than 0.10.

The monomers and/or oligomers provided in method step a) may be neutralized at a degree of neutralization from 40 to 95 mol %.

The optional co-monomers may be provided at less than 30 weight-%, or less than 20 weight-%, or less than 15 weight-% or less than 10 weight-%, or less than 5 weight-%, or even less than 2 weight-% based on the total weight of the polymerizable acrylic acid monomers and/or polymerizable acrylic acid oligomers.

The invention also relates to superabsorbent polymer material comprising cross-linked polyacrylic acid and salts thereof, the superabsorbent polymer material comprising polyacrylic acid as internal cross-linkers of the network. The polyacrylic acid internal cross-linker may be the only crosslinker of the SAP material (apart from an optional surface crosslinker). Such SAP materials can be obtained by the method of the present invention.

Absorbent articles comprising the superabsorbent polymer material of the invention are also provided.

The superabsorbent polymer material may be at least partially neutralized, preferably from 50% to 95% neutralized.

The superabsorbent polymer material may have an EFFC of at least 25 g/g.

### BRIEF DESCRIPTION OF THE DRAWINGS

FIG. 1 is a top view of an exemplary absorbent article in the form of a diaper, which may comprise the agglomerated superabsorbent polymer particles of the present invention, with some layers partially removed.

FIG. 2 is a transversal cross-section of the diaper of FIG. 1.

FIG. 3 is a partial cross-sectional side view of a suitable permeability measurement system for conducting the Urine Permeability Measurement Test.

FIG. 4 is a cross-sectional side view of a piston/cylinder assembly for use in conducting the Urine Permeability Measurement Test.

FIG. 5 is a top view of a piston head suitable for use in the piston/cylinder assembly shown in FIG. 4.

FIG. 6 is a cross-sectional side view of the piston/cylinder assembly of FIG. 4 placed on fritted disc for the swelling phase.

### DETAILED DESCRIPTION OF THE INVENTION

### Definitions

“Absorbent article” refers to devices that absorb and contain body exudates, particularly urine and other water-containing liquids, and, more specifically, refers to devices that are placed against or in proximity to the body of the wearer to absorb and contain the various exudates discharged from the body. Absorbent articles may include diapers (diapers for babies and infants and diapers to address adult incontinence), pants (pants for babies and infants and pants to address adult incontinence), disposable absorbent inserts for diapers and pants having a re-usable outer cover), feminine care absorbent articles such as sanitary napkins or pantiliners, breast pads, care mats, bibs, wipes, and the like. As used herein, the term “exudates” includes, but is not limited to, urine, blood, vaginal discharges, breast milk, sweat and fecal matter, Preferred absorbent articles of the present invention are disposable absorbent articles, more preferably disposable diapers, disposable pants and disposable absorbent inserts.

“Absorbent core” is used herein to refer to a structure disposed between a topsheet and backsheet of an absorbent article for absorbing and containing liquid received by the absorbent article.

“Airfelt” is used herein to refer to comminuted wood pulp, which is a form of cellulosic fiber,

“Base polymer particles” as used herein, refers to SAP particles, which have not undergone any surface treatment, such as surface cross-linking and/or surface coating, after having been polymerized and comminuted into superabsorbent polymer particles.

Generally, base polymer particles have higher capacity and lower permeability compared to surface treated SAP particles.

As used herein, the term “degradation” refers to the conversion of SAP into soluble PAA polymers via the actions of de-polymerization, de-crosslinking, molecular backbone breaking, or any combination thereof.

“Disposable” is used in its ordinary sense to mean an article that is disposed or discarded after a limited number of usage events over varying lengths of time, for example, less than 10 events, less than 5 events, or less than 2 events. If the disposable absorbent article is a diaper, a pant, absorbent insert, sanitary napkin, sanitary pad or wet wipe for personal hygiene use, the disposable absorbent article is most often intended to be disposed after single use.

“Diaper” and “pant” refers to an absorbent article generally worn by babies, infants and incontinent persons about the lower torso so as to encircle the waist and legs of the wearer and that is specifically adapted to receive and contain urinary and fecal waste. In a pant, as used herein, the longitudinal edges of the first and second waist region are attached to each other to a pre-form waist opening and leg openings. A pant is placed in position on the wearer by inserting the wearer's legs into the leg openings and sliding the pant absorbent article into position about the wearer's lower torso. A pant may be pre-formed by any suitable technique including, but not limited to, joining together portions of the absorbent article using refastenable and/or non-refastenable bonds (e.g., seam, weld, adhesive, cohesive bond, fastener, etc.). A pant may be pre-formed anywhere along the circumference of the article (e.g., side fastened, front waist fastened). In a diaper, the waist opening and leg openings are only formed when the diaper is applied onto a wearer by (releasable) attaching the longitudinal edges of the first and second waist region to each other on both sides by a suitable fastening system.

“Superabsorbent polymer material” (“SAP material”) is used herein to refer to crosslinked polymeric materials that can absorb at least 10 times their weight of an aqueous 0.9% saline solution as measured using the Centrifuge Retention Capacity test set out below. Superabsorbent polymer material of the present invention is made of polyacrylic acid polymers.

“Superabsorbent polymer particles” (“SAP particles”) is used herein to refer to superabsorbent polymer material that is in particulate form so as to be flowable in the dry state.

“Pre-existing superabsorbent polymer material” (“pre-existing SAP material”) is used herein to refer to SAP material that is not within the scope of the invention but that is material that has been degraded to obtain s-PAA polymers which can be used for the present invention.

“Soluble polyacrylic acid polymers” (hereinafter abbreviated as “s-PAA polymers”) are polymers that are soluble in aqueous solutions. They are not cross-linked to be above the gel-point. The “gel point” is an abrupt change in the viscosity of a solution containing a polymer. At the gel point, a solution undergoes gelation, leading to a gel formation, as reflected in a loss in fluidity and the formation of a 3D network (i.e. cross-linked polymer chains).

“Polymer backbone” is the longest series of covalently bonded atoms that together create the continuous chain of the molecule. Polyacrylic acid polymers have a carbon backbone. As used herein, the polymer backbone can be unbranched (containing one linear chain) or branched (containing multiple chains).

“% wt”, “% w” “weight-%”, and “wt %” are used herein interchangeably and all mean “percent weight”.

Method of making the SAP material comprising s-PAA polymers

The invention relates to a method of making superabsorbent polymer material. The method comprises the steps of
a) providing an aqueous solution of polymerizable acrylic acid monomers and/or polymerizable acrylic acid oligomers, optionally neutralizing at least some of the polymerizable acrylic acid monomers and/or polymerizable acrylic acid oligomers;
b) optionally providing one or more ethylenically unsatured co-monomers, optionally neutralizing at least some of the ethylenically unsatured co-monomer of step b);
c) optionally providing one or more crosslinker(s);
d) providing one or more initiator(s);
e) providing soluble polyacrylic acid polymers, wherein the soluble polyacrylic acid polymers have a molar percent of carbon-to-carbon double bonds of at least 0.02, preferably at least 0.04, more preferably at least 0.05, still more preferably at least 0.08, and even more preferably at least 0.1;
f) mixing the aqueous solution of monomers, oligomers, co-monomers, crosslinkers and initiators and soluble polyacrylic acid polymers provided in steps a) to e); and
g) polymerizing the mixture obtained in step f) to obtain a superabsorbent polymer.

Having s-PAA polymers wherein the soluble polyacrylic acid polymers have a molar percent of carbon-to-carbon double bonds of at least 0.02 ensures that the s-PAA polymers have a sufficiently high number of carbon-to-carbon double bonds such that they can readily polymerize into the polymeric network of the SAP material obtained by the method. Not every single s-PAA polymer molecule may actually contain a carbon-to-carbon double bond, however, as long as a significant number of carbon-to-carbon double bonds can be determined (in accordance with the method set out below), the s-PAA polymers can be covalently bound into the polymer network of the SAP material obtained by the method, thus significantly reducing the amount of extractables. The higher the molar percent of carbon-to-carbon double bonds, the higher is the number of such double bonds in the s-PAA polymer.

When providing the s-PAA polymers in method step e) the s-PAA polymers may be provided to the aqueous solution in dry form (as powder) or may be provided as aqueous solution. As s-PAA polymers are often hard to dissolve, it may indeed be beneficial to provide the s-PAA polymers as aqueous solution. Moreover, if the s-PAA polymers are obtained from degradation of pre-existing post-consumer recycled SAP material, the degradation product (i.e. the s-PAA polymers) would most likely be an aqueous solution, so drying and re-dissolving the s-PAA polymers would be time-and energy-consuming.

The s-PAA polymers may be provided in step e) at a weight-percent of at least 3 weight-%, preferably at least 5 weight-% and more preferably at least 10 weight-% based on the total weight of the soluble polyacrylic acid polymers provided in step e) and the monomers, oligomers, co-monomers, crosslinkers and initiators provided in steps a) to d). The weight-percent of the s-PAA polymers based on the total weight of the soluble polyacrylic acid polymers provided in step e) and the monomers, oligomers, co-monomers, crosslinkers and initiators provided in steps a) to d), is also referred to as add-on level herein below.

The s-PAA polymers may be provided in step e) at a weight-percent of up to 70 weight-%, or up to 60.0 weight-%, or up to 50.0 weight-%, or up to 40.0 weight-%, or up to 30.0 weight-%, or up to 25.0 weight-% based on the total weight of the soluble polyacrylic acid polymers provided in step e) and the monomers, oligomers, co-monomers, crosslinkers and initiators provided in steps a) to d).

As it can be assumed that all components provided in steps a) to e) react in the polymerization, the weight-% of step e) is the same as the weight-% of s-PAA polymers in the superabsorbent polymer material obtained by the method.

The SAP material may be dried after polymerization. The SAP material may also be comminuted to obtain SAP particles. Comminuting may be done subsequent to drying or may be done prior to drying (e.g. by so-called wet grinding).

The optional ethylenically unsaturated co-monomers provided in method step b) may be water-soluble, i.e. their solubility in water at 23° C. is typically at least 1 g/100 g of water, preferably at least 5 g/100 g of water, more preferably at least 25 g/100 g of water and most preferably at least 35 g/100 g of water.

Suitable ethylenically unsatured co-monomers optionally provided in method step b) are, for example, ethylenically unsaturated carboxylic acids, such as methacrylic acid and itaconic acid.

Further suitable ethylenically unsatured co-monomers provided in method step b) are, for example, ethylenically unsaturated sulfonic acids, such as styrenesulfonic acid.

Other ethylenically unsaturated co-monomers that may be added in combination with acrylic acid, methacrylic acid, itaconic acid or ethylenically unsaturated sulfonic acids, are styrenesulfonic acid copolymerizable with the ethylenically unsaturated monomers provided in method step a) are, for example, acrylamide, methacrylamide, hydroxyethyl acrylate, hydroxyethyl methacrylate, dimethylaminoethyl methacrylate, dimethylaminoethyl acrylate, dimethylaminopropyl acrylate, diethylaminopropyl acrylate, dimethylaminoethyl methacrylate, and/or diethylaminoethyl methacrylate.

The acid groups of the monomers a) and/or co-comonomers b) may have been partly neutralized. The neutralization can be conducted at the monomer stage. This can typically be accomplished by mixing in the neutralizing agent as an aqueous solution or else preferably as a solid. The degree of neutralization may preferably be from 40 to 95 mol %, more preferably from 40 to 80 mol % and most preferably from 50 to 75 mol %. A customary neutralizing agent can be used, preferably alkali metal hydroxides, alkali metal oxides, alkali metal carbonates or alkali metal hydrogencarbonates and also mixtures thereof. Instead of alkali metal salts, it is also possible to use ammonium salts. Particularly preferred alkali metals are sodium and potassium, but very particular preference is given to sodium hydroxide, sodium carbonate or sodium hydrogencarbonate and also mixtures thereof.

Suitable crosslinkers optionally provided in method step b) are compounds having at least two groups suitable for crosslinking. Such groups are, for example, ethylenically unsaturated groups which can be polymerized free-radically into the polymer chain, and functional groups which can form covalent bonds with the acid groups of the monomer provided in method step a) and/or with the co-monomers provided in method step b). In addition, polyvalent metal salts which can form coordinate bonds with at least two acid groups of the monomer provided in method step a) are also suitable as crosslinkers.

The optional crosslinkers provided in method step c) are preferably compounds having at least two polymerizable groups which can be polymerized free-radically into the polymer network. Suitable crosslinkers provided in method step b) are, for example, methylenebisacrylamide, ethylene glycol dimethacrylate, diethylene glycol diacrylate, polyethylene glycol diacrylate, allyl methacrylate, trimethylolpropane triacrylate, triallylamine, tetraallylammonium chloride, tetraallyloxyethane, or mixed acrylates which, as well as acrylate groups, comprise further ethylenically unsaturated groups.

The amount of crosslinker provided in method step c) is preferably 0.0001% to 0.5% by weight, more preferably 0.001% to 0.2% by weight and most preferably 0.01% to 0.1% by weight, based on the total weight of the un-neutralized monomer provided in method step a) and un-neutralized co-comonomers provided in step b).

The optional crosslinker provided in step c) is different from the s-PAA polymers provided in step e). Hence, the optional crosslinker provided in step c) is not a polyacrylic acid polymer having carbon-to-carbon double bonds.

As the s-PAA polymers having carbon-to-carbon double bonds provided in step e) function as crosslinkers, the provision of additional crosslinkers is optional. If additional crosslinkers are provided, the amount (in weight-%) can be kept relatively low.

Initiators provided in method step d) may be all compounds which generate free radicals under the polymerization conditions, for example thermal initiators, redox initiators or photoinitiators.

Suitable redox initiators are potassium peroxodisulfate or sodium peroxodisulfate/ascorbic acid, hydrogen peroxide/ascorbic acid, potassium peroxodisulfate or sodium peroxodisulfate/sodium bisulfite and hydrogen peroxide/sodium bisulfite. Preferably, mixtures of thermal initiators and redox initiators are used, such as potassium peroxodisulfate or sodium peroxodisulfate/hydrogen peroxide/ascorbic acid. The reducing component used is, however, preferably a mixture of the sodium salt of 2-hydroxy-2-sulfinatoacetic acid, the disodium salt of 2-hydroxy-2-sulfonatoacetic acid and sodium bisulfite. Such mixtures can be obtained as Brüggolite® FF6 and Brüggolite® FF7 (Brüggemann Chemicals; Heilbronn; Germany).

Suitable thermal initiators are especially azo initiators, such as 2,2′-azobis[2-(2-imidazolin-2-yl) propane] dihydrochloride and 2,2′-azobis[2-(5-methyl-2-imidazolin-2-yl)propane] dihydrochloride, 2,2′-azobis(2-amidinopropane) dihydrochloride, 4,4″-azobis(4-cyanopentanoic acid), 4,4′ and the sodium salts thereof, 2,2′-azobis[2-methyl-N-(2-hydroxyethyl)propionamide] and 2,2′-azobis(imino-1-pyrrolidino-2-ethylpropane) dihydrochloride.

Suitable photoinitiators are, for example, 2-hydroxy-2-methylpropiophenone and 1-[4-(2-hydroxyethoxy)phenyl]-2-hydroxy-2-methyl-1-propan-1-one.

Mixing and polymerization of method steps f) and g) may be done in a kneading reactor or belt reactor. In the kneading reactor, the polymer gel formed in the polymerization is comminuted continuously by, for example, contra-rotatory stirrer shafts. Polymerization in a belt reactor is also well known in the art. Polymerization in a belt reactor forms a polymer gel which has to be comminuted in a further process step, for example in an extruder or kneader.

The s-PAA polymers having carbon-to-carbon double bonds as are provided in step e) may be obtained from pre-existing recycled post-consumer superabsorbent polymer material or obtained from pre-existing recycled post-industrial superabsorbent polymer material. Thus, the method may comprise the further step of a1) obtaining the s-PAA polymers from pre-existing recycled post-consumer superabsorbent polymer material or from pre-existing recycled post-industrial superabsorbent polymer material. These s-PAA polymers may be obtained by chemical degradation of the pre-existing recycled post-consumer superabsorbent polymer material. Step a1) may be carried out prior to step b).

The chemical degradation may be done with an oxidative water-soluble salt comprising at least one cation and at least one anion. The at least one anion may be selected from the group consisting of: peroxydisulfate, peroxymonosulfate, peroxydicarbonate, peroxydiphosphate, peroxydiborate and mixtures and combinations thereof.

Instead of chemical degradation with an oxidative water-soluble salt, the chemical degradation may be mediated by redox couples. It is well known in the art that primary radicals may be generated via redox couple, allowing for more controlled radical flux at lower temperature than thermal decomposition alone. The redox couples may be selected from the group consisting of sodium peroxodisulfate/ascorbic acid; hydrogen peroxide/ascorbic acid; potassium peroxodisulfate/sodium bisulfite; sodium peroxodisulfate/sodium bisulfite; hydrogen peroxide/sodium bisulfite; potassium peroxodisulfate/ascorbic acid and combinations thereof.

If the s-PAA polymers having carbon-to-carbon double bonds are derived from degradation of pre-existing SAP material, such as pre-existing SAP particles, the pre-existing SAP material can be pre-existing virgin SAP material, pre-existing post-consumer recycled SAP material, pre-existing post-industrial recycled SAP material, or a combination of those materials. “Post-consumer recycled SAP material”, as used herein, refer to pre-existing SAP material which has been comprised by an absorbent article and the absorbent article has been used by a consumer (e.g. worn by an incontinent user). After use, the absorbent article is recycled, and the pre-existing post-consumer recycled SAP material is isolated from the absorbent article and is degraded into s-PAA polymers. “Post-industrial recycled SAP material”, as used herein, refer to pre-existing SAP material which may or may not have been comprised by an absorbent article. The post-industrial recycled SAP material has not been previously used, e.g. it was not comprised by an absorbent article which has been used by a consumer. Instead, the post-industrial recycled SAP material may be derived from absorbent articles which have been sorted out during production, e.g. because they were defective. The post-industrial recycled SAP material which was not comprised by absorbent articles may have been sorted out during production of the previous SAP material, e.g. because it did not meet the required performance targets (such as particle size distribution (PSD), capacity, whiteness or the like).

The s-PAA polymers provided in step e) may have a weight average molecular weight Mw of at least 50 kDa, or at least 100 kDa, or at least 120 kDa, or at least 150 kDa, or at least 200 kDa.

The s-PAA polymers provided in step e) may have a weight average molecular weight Mw of not more than 3 MDa, or not more than 2 MDa, or not more than 1.5 MDa, or not more than 1 MDa.

The SAP material obtained by the method may have an amount of extractables of less than 15.0 weight-%, or less than 13 weight-%, or less than 12 weight-% based on the total weight of the superabsorbent polymer material.

The SAP material obtained by the method may have a capacity measured as Centrifuge Retention Capacity (CRC) in accordance the test method set out herein of at least 20 g/g. The SAP materials obtained by the method may have a ratio of amount of the difference between extractables (weight-%) and s-PAA polymer add-on (% wt), to capacity (g/g) of less than 0.15, or less than 0.12, or less than 0.10.

The method may comprise a further step of surface crosslinking the SAP particles obtained by the method (wherein the SAP particles are obtained by additional method steps of drying the SAP material and comminuting the SAP material). Surface cross-linking may be performed in such a way that a solution, such as an aqueous solution, of the surface crosslinker is sprayed onto the dried SAP particles. After the spray application, the surface crosslinker-coated polymer particles are thermally surface crosslinked.

Spray application of a solution of the surface crosslinker onto the SAP particles is preferably performed in mixers with moving mixing tools, such as screw mixers, disk mixers and paddle mixers.

Superabsorbent polymer material comprising soluble polyacrylic acid polymers

Superabsorbent polymer material of the present invention comprises cross-linked polyacrylic acid and salts thereof, the superabsorbent polymer material comprising polyacrylic acid as internal cross-linkers of the network. The polyacrylic acid may be the only internal cross-linkers of the SAP material.

If the polyacrylic acid is the only internal crosslinker, this shows that no additional crosslinkers have been applied to the method of making the SAP material.

The SAP material may be in the form of superabsorbent polymer particles. The SAP particles may be surface cross-linked. The SAP may be coated-either in addition to being surface cross-linked or instead of being surface cross-linked.

The SAP material of the invention may have an amount of extractables of less than. 15% weight, or less than 13% weight or less than 12% weight. The amount of extractables generally increases if the capacity of the SAP material increases.

The SAP material of the present invention may have a capacity of at least 20 g/g, as measured in accordance with the Centrifuge Retention Capacity (CRC) method set out below.

The SAP material of the present invention may have an EFFC value of at least 25 g/g, or at least 25 g/g. The EFFC value combines the capacity (CRC) and the Absorption Against Pressure (AAP) performance of the SAP material as

$$EFFC = ( CRC + AAP ) / 2$$

If the SAP material is in the form of SAP particles, the SAP particles may be of numerous shapes. The term “particles” refers to granules, fibers, flakes, spheres, powders, platelets and other shapes and forms known to persons skilled in the art of SAP particles. In some embodiments, the SAP particles can be in the shape of fibers, i.e. elongated, acicular superabsorbent polymer particles. In those embodiments, the SAP fibers have a minor dimension (i.e. diameter of the fiber) of less than about 1 mm, usually less than about 500 μm, and preferably less than 250 μm down to 50 μm. The length of the fibers is preferably about 3 mm to about 100 mm. The fibers can also be in the form of a long filament that can be woven.

Alternatively, the SAP particles of the present invention are spherical-like particles. According to the present invention and in contrast to fibers, “spherical-like particles” have a longest and a smallest dimension with a particulate ratio of longest to smallest particle dimension in the range of 1-5, where a value of 1 would equate a perfectly spherical particle and 5 would allow for some deviation from such a spherical particle. In such embodiments, the SAP particles may have a particle size of less than 850 μm, or from 50 to 850 μm, preferably from 100 to 500 μm, more preferably from 150 to 300 μm, as measured according to EDANA method WSP 220.2-05. SAP particles having a relatively low particle size help to increase the surface area which is in contact with liquid exudates and therefore support fast absorption of liquid exudates.

The superabsorbent polymer material may be partially neutralized, e.g. by polymerizing the acrylic acid monomers at 40 mol % to 95 mol % neutralization, or at 50 mol % to 80 mol % neutralization, or at 55 mol % to75 mol % neutralization. The superabsorbent polymer material may alternatively, or in addition, be neutralized after polymerization, such that the total degree of neutralization is 40-95 mol %, or 50-80 mol %, or 55-75 mol %.

The term “surface” describes the outer-facing boundaries of the particle. For porous SAP particles, exposed internal surfaces may also belong to the surface. The term “surface cross-linked SAP particle” refers to an SAP particle having its molecular chains present in the vicinity of the particle surface cross-linked by a compound referred to as surface cross-linker. The surface cross-linker is applied to the surface of the particle. In a surface cross-linked SAP particle, the level of cross-links in the vicinity of the surface of the SAP particle is generally higher than the level of cross-links in the interior of the SAP.

Commonly applied surface cross-linkers are thermally activatable surface cross-linkers. The term “thermally activatable surface cross-linkers” refers to surface cross-linkers, which only react upon exposure to increased temperatures, typically around 150° C. Thermally activatable surface cross-linkers known in the prior art are e.g. di- or polyfunctional agents that are capable of building additional cross-links between the polymer chains of the SAPs. Examples of thermally activatable surface cross-linkers include but are not limited to: di- or polyhydric alcohols, or derivatives thereof, capable of forming di-or poly-hydric alcohols, alkylene carbonates, ketales, and di- or polyglycidlyethers, haloepoxy compounds, polyaldehydes, polyoles and polyamines. The cross-linking is based on a re-action between the functional groups comprised by the polymer, for example, an esterification reaction between a carboxyl group (comprised by the polymer) and a hydroxyl group (comprised by the surface cross-linker). As typically a relatively large fraction of the carboxyl groups of the polymer chain is neutralized prior to the polymerization step, commonly only few carboxyl groups are available for this surface cross-linking process known in the art. E.g. in a 70% percent neutralized polymer only 3 out of 10 carboxylic groups are available for covalent surface cross-linking.

The surface of the SAP particles may be coated, either instead of being surface cross-linked or, more preferably, in addition to being surface crosslinked (wherein coating is carried out after surface cross-linking). The coating makes the surface sticky so that SAP particles cannot rearrange (so they cannot block voids) easily upon wetting.

For example, the SAP particles may be coated with a cationic polymer. Preferred cationic polymers can include polyamine or polyimine materials which are reactive with at least one component included in body fluids, especially in urine. Preferred polyamine materials are selected from the group consisting of (1) polymers having primary amine groups (e.g., polyvinylamine, polyallyl amine); (2) polymers having secondary amine groups (e.g., polyethyleneimine); and (3) polymers having tertiary amine groups (e.g., poly N, N-dimethylalkyl amine). Practical examples of the cationic polymer are, for example, polyethyleneimine, a modified polyethyleneimine which is crosslinked by epihalohydrine in a range soluble in water, polyamine, a modified polyamidoamine by graft of ethyleneimine, polyetheramine, polyvinylamine, polyalkylamine, polyamidopolyamine, and polyallylamine.

A cationic polymer coated on the surface of the SAP particle may have a weight-average molecular weight Mw of at least 500 Da, more preferably 5,000 Da, most preferably 10,000 Da or more. Cationic polymers having a weight-average molecular weight of more than 500 or more are not limited to polymers showing a single maximum value (a peak) in a molecular weight analysis by gel permeation chromatography, and polymers having a weight-average molecular weight of 500 or more may be used even if it exhibits a plural maximum value (peaks).

A preferable amount of the cationic polymer is in a range of from about 0.05 to 20 parts by weight against 100 parts by weight of the superabsorbent polymer particle, more preferably from about 0.3 to 10 parts by weight, and most preferably from about 0.5 to 5 parts by weight.

### Absorbent Articles

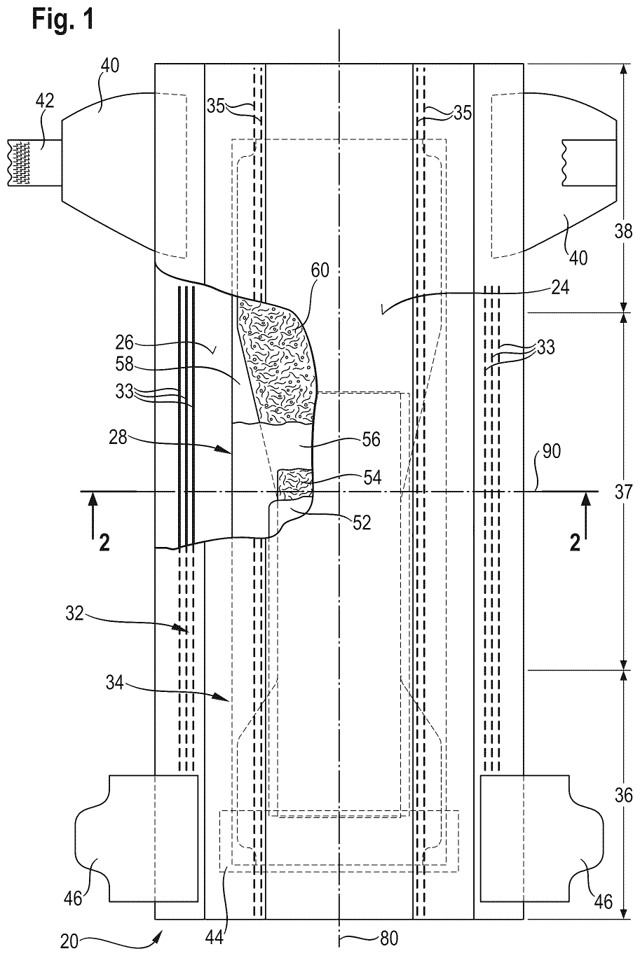

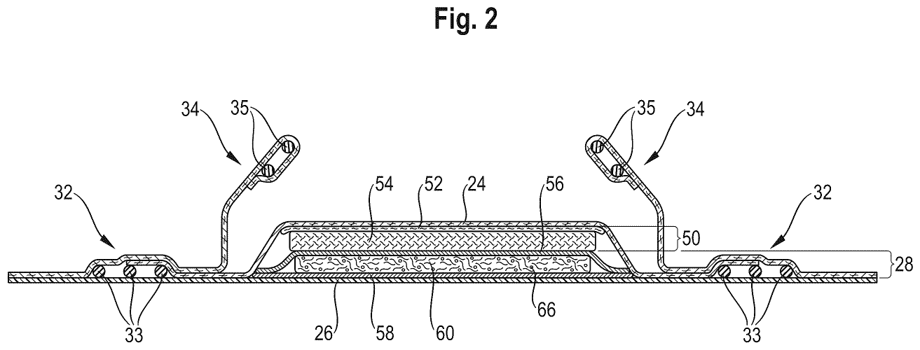

A typical disposable absorbent article, in which SAP material of the present invention can be used, is placed against or in proximity to the body of the wearer to absorb and contain the various exudates discharged from the body and is represented in FIGS. 1 and 2 in the form of a diaper 20.

In more details, FIG. 1 is a plan view of an exemplary diaper 20, in a flat-out state, with portions of the diaper being cut-away to more clearly show the construction of the diaper 20. This diaper 20 is shown for illustration purpose only as the SAP material of the present invention may be comprised in a wide variety of diapers or other absorbent articles.

As shown in FIGS. 1 and 2, the absorbent article, here a diaper, can comprise a liquid pervious topsheet 24, a liquid impervious backsheet 26, an absorbent core 28 which is positioned between f the topsheet 24 and the backsheet 26. The absorbent core 28 can absorb and contain liquid received by the absorbent article and may comprise absorbent materials 60, such as the SAP material of the present invention 66 and/or cellulose fibers, as well as other absorbent and non-absorbent materials commonly used in absorbent articles (e.g. thermoplastic adhesives immobilizing the SAP particles). The absorbent material and non-absorbent material may be wrapped within a substrate (e.g. one or more nonwovens, tissues etc.) such as by an upper core cover layer 56 facing towards the topsheet and a lower cover layer 58 facing towards the backsheet. Such upper and lower core cover layers may be made of nonwovens, tissues or the like and may be attached to each other continuously or discontinuously, e.g. along their perimeter.

The absorbent core may comprise one or more substrate layer(s) (such as nonwoven webs or paper tissue), SAP material (such as SAP particles) disposed on the one or more substrate layers, and a thermoplastic composition typically disposed on the SAP material (such as SAP particles). Typically, the thermoplastic composition is a thermoplastic adhesive material. In one embodiment, the thermoplastic adhesive material forms a fibrous layer which is at least partially in contact with the SAP material (such as SAP particles) on the one or more substrate layers and partially in contact with the one or more substrate layers. Auxiliary adhesive might be deposited on the one or more substrate layers before application of the SAP material (such as SAP particles) for enhancing adhesion of the SAP material (e.g. SAP particles) and/or of the thermoplastic adhesive material to the respective substrate layer(s). The absorbent core may also include one or more cover layer(s) such that the SAP material (e.g. SAP particles) are comprised between the one or more substrate layer(s) and the one or more cover layer(s). The one or more substrate layer(s) and the cover layer(s) may comprise or consist of a nonwoven web. The absorbent core may further comprise odor control compounds.

The absorbent core may consist essentially of the one or more substrate layer(s), the SAP material (e.g. SAP particles), the thermoplastic composition, optionally the auxiliary adhesive, optionally the cover layer(s), and optionally odor control compounds.

The absorbent core may also comprise a mixture of SAP particles and airfelt, which may be enwrapped within one or more substrate layers, such as nonwoven webs or paper tissue. Such absorbent cores may comprise from 30% to 95%, or from 50% to 95% of SAP particles by weight of the absorbent material and may comprise from 5% to 70%, or from 5% to 50% of airfelt by weight of the absorbent material (for these percentages, any enwrapping substrate layers are not considered as absorbent material). The absorbent core may also be free of airfelt and may comprise 100% of SAP particles by weight of the absorbent material.

The absorbent core may comprise mixtures or combinations of the SAP material of the present invention and other SAP materials (such as other SAP particles, and/or SAP foams). For example, the absorbent core may comprise at least 50%, or at least 60%, or at least 70%, or at least 80%, or at least 90% or 100% of SAP material by weight of the absorbent material, wherein the SAP material comprise at least 10%, or at least 20% or at least 30% or at least 50%, or at least 75%, or at least 90%, or 100% by weight of the SAP material of the present invention based on the total weight of SAP material in the absorbent core.

The absorbent articles of the invention, especially diapers and pants, may comprise an acquisition layer 52, a distribution layer 54, or combination of both (all herein collectively referred to as acquisition-distribution system “ADS” 50). The function of the ADS 50 is typically to quickly acquire the fluid and distribute it to the absorbent core in an efficient manner. The ADS may comprise one, two or more layers. In the examples below, the ADS 50 comprises two layers: a distribution layer 54 and an acquisition layer 52 disposed between the absorbent core and the topsheet.

The ADS may be free of SAP material. The prior art discloses many types of acquisition-distribution systems, see for example WO2000/59430, WO95/10996, U.S. Pat. No. 5,700,254, WO02/067809. However, the SAP material of the present invention may also be comprised by the ADS.

The function of a distribution layer 54 is to spread the insulting fluid liquid over a larger surface within the article so that the absorbent capacity of the absorbent core can be more efficiently used. Distribution layers may be made of a nonwoven material based on synthetic or cellulosic fibers and having a relatively low density. The distribution layer may typically have an average basis weight of from 30 to 400 g/m², in particular from 80 to 300 g/m².

The distribution layer may for example comprise at least 50%, or 60%, or 70%, or 80%, or 90%, or 100% by weight of cross-linked cellulose fibers. The cross-linked cellulosic fibers may be crimped, twisted, or curled, or a combination thereof including crimped, twisted, and curled. The cross-linked cellulosic fibers provide higher resilience and therefore higher resistance to the first absorbent layer against the compression in the product packaging or in use conditions, e.g. under baby weight. This provides the core with a relatively high void volume, permeability and liquid absorption, and hence reduced leakage and improved dryness.

The absorbent article 20 may further comprise an acquisition layer 52, whose function is to quickly acquire the fluid away from the topsheet so as to provide a good dryness for the wearer. The acquisition layer 52 is typically placed directly under the topsheet and below the distribution layer. The acquisition layer may typically be or comprise a non-woven material, for example a SMS or SMMS material, comprising a spunbonded, a melt-blown and a further spunbonded layer or alternatively a carded chemical-bonded nonwoven. The non-woven material may in particular be latex bonded. Exemplary upper acquisition layers 52 are disclosed in U.S. Pat. No. 7,786,341. Carded, resin-bonded nonwovens may be used, in particular where the fibers used are solid round or round and hollow PET staple fibers (such as a 50/50 or 40/60 mix of 6 denier and 9 denier fibers). An exemplary binder is a butadiene/styrene latex.

The acquisition layer 52 may be stabilized by a latex binder, for example a styrene-butadiene latex binder (SB latex). Processes for obtaining such lattices are known, for example, from EP 149 880 (Kwok) and US 2003/0105190 (Diehl et al.). The binder may be present in the acquisition layer 52 in excess of 12%, 14% or 16% by weight, but may be present by not more than 30%, or not more than 25% by weight of the acquisition layer. SB latex is available under the trade name GENFLO™ 3160 (OMNOVA Solutions Inc.; Akron, Ohio).

The diaper may also comprise elasticized leg cuffs 32 and barrier leg cuffs 34, which provide improved containment of liquids and other body exudates especially in the area of the leg openings. Usually each leg cuffs 32 and barrier cuffs 34 will comprise one or more elastic string 33 and 35, represented in exaggerated form on FIGS. 1 and 2. Moreover, the diaper 20 may comprise other features such as back ears 40, front ears 46 and/or barrier cuffs 34 attached to form the composite diaper structure. The diaper may further comprise a fastening system, such as an adhesive fastening system or a mechanical fastening system (e.g. a hook and loop fastening system), which can comprise tape tabs 42, such as adhesive tape tabs or tape tabs comprising hook elements, cooperating with a landing zone 44 (e.g. a nonwoven web providing loops in a hook and loop fastening system). Further, the diaper may comprise other elements, such as a back elastic waist feature and a front elastic waist feature, side panels or a lotion application.

The diaper 20 as shown in FIGS. 1 and 2 can be notionally divided in a first waist region 36, a second waist region 38 opposed to the first waist region 36 and a crotch region 37 located between the first waist region 36 and the second waist region 38. The longitudinal centerline 80 is the imaginary line separating the diaper along its length in two equal halves. The transversal centerline 90 is the imagery line perpendicular to the longitudinal line 80 in the plane of the flattened out diaper and going through the middle of the length of the diaper. The periphery of the diaper 20 is defined by the outer edges of the diaper 20. The longitudinal edges of the diaper may run generally parallel to the longitudinal centerline 80 of the diaper 20 and the end edges run between the longitudinal edges generally parallel to the transversal centerline 90 of the diaper 20.

### Bio-Based Materials

Absorbent articles comprising the SAP material of the present invention, may comprise a bio-based content value from about 10% to about 100% using ASTM D6866-10, method B, or from about 25% to about 75%, or from about 50% to about 60%.

The various components of the absorbent article, such as the topsheet, the backsheet, the fasteners, the ADS, the back ears, the outer cover nonwoven, an elastic laminate (such as the elastic laminate forming a belt of an absorbent aricle), or any other component, may comprise a bio-based content value from about 10% to about 100% using ASTM D6866-10, method B, or from about 25% to about 75%, or from about 50% to about 60%.

In order to apply the methodology of ASTM D6866-10 to determine the bio-based content of a single component material (i.e. a nonwoven), that material is isolated and cleaned such that the resulting specimen reflects the constituent starting material as closely as possible. For example, if a component needs to be deconstructed (e.g. removal elastic strands removed from a laminate formed of one or more nonwovens and elastic strands) the nonwoven is washed with an appropriate solvent so as to remove any residual adhesive present. In order to apply the methodology of ASTM D6866-10 to an sample assembly of two or more materials of differing or unknown compositions, the sample is homogenized by grinding the material into particulate form (with particle size of about 20 mesh or smaller) using known grinding methods (such as with a Wiley grinding mill). A representative specimen of suitable mass is then taken from the resulting sample of randomly mixed particles.

### Validation of Polymers Derived from Renewable Resources

A suitable validation technique is through 14C analysis. A small amount of the carbon dioxide in the atmosphere is radioactive. This 14C carbon dioxide is created when nitrogen is struck by an ultra-violet light produced neutron, causing the nitrogen to lose a proton and form carbon of molecular weight 14 which is immediately oxidized to carbon dioxide. This radioactive isotope represents a small but measurable fraction of atmospheric carbon. Atmospheric carbon dioxide is cycled by green plants to make organic molecules during photosynthesis. The cycle is completed when the green plants or other forms of life metabolize the organic molecules, thereby producing carbon dioxide which is released back to the atmosphere. Virtually all forms of life on Earth depend on this green plant production of organic molecules to grow and reproduce. Therefore, the 14C that exists in the atmosphere becomes part of all life forms, and their biological products. In contrast, fossil fuel based carbon does not have the signature radiocarbon ratio of atmospheric carbon dioxide.

Assessment of the renewably based carbon in a material can be performed through standard test methods. Using radiocarbon and isotope ratio mass spectrometry analysis, the bio-based content of materials can be determined. ASTM International, formally known as the American Society for Testing and Materials, has established a standard method for assessing the bio-based content of materials. The ASTM method is designated ASTM D6866-10.

The application of ASTM D6866-10 to derive a “bio-based content” is built on the same concepts as radiocarbon dating, but without use of the age equations. The analysis is performed by deriving a ratio of the amount of organic radiocarbon (14C) in an unknown sample to that of a modern reference standard. The ratio is reported as a percentage with the units “pMC” (percent modern carbon).

The modern reference standard used in radiocarbon dating is a NIST (National Institute of Standards and Technology) standard with a known radiocarbon content equivalent approximately to the year AD 1950. AD 1950 was chosen since it represented a time prior to thermo-nuclear weapons testing which introduced large amounts of excess radiocarbon into the atmosphere with each explosion (termed “bomb carbon”). The AD 1950 reference represents 100 pMC.

“Bomb carbon” in the atmosphere reached almost twice normal levels in 1963 at the peak of testing and prior to the treaty halting the testing. Its distribution within the atmosphere has been approximated since its appearance, showing values that are greater than 100 pMC for plants and animals living since AD 1950. It's gradually decreased over time with today's value being near 107.5 pMC. This means that a fresh biomass material such as corn could give a radiocarbon signature near 107.5 pMC.

Combining fossil carbon with present day carbon into a material will result in a dilution of the present day pMC content. By presuming 107.5 pMC represents present day biomass materials and 0 pMC represents petroleum derivatives, the measured pMC value for that material will reflect the proportions of the two component types. A material derived 100% from present day soybeans would give a radiocarbon signature near 107.5 pMC. If that material was diluted with 50% petroleum derivatives, for example, it would give a radiocarbon signature near 54 pMC (assuming the petroleum derivatives have the same percentage of carbon as the soybeans).

A biomass content result is derived by assigning 100% equal to 107.5 pMC and 0% equal to 0 pMC. In this regard, a sample measuring 99 pMC will give an equivalent bio-based content value of 92%.

Assessment of the materials described herein can be done in accordance with ASTM D6866. The mean values quoted in this report encompasses an absolute range of 6% (plus and minus 3% on either side of the bio-based content value) to account for variations in end-component radiocarbon signatures. It is presumed that all materials are present day or fossil in origin and that the desired result is the amount of biobased component “present” in the material, not the amount of biobased material “used” in the manufacturing process.

### Test Methods

NMR Alkene Content Method (Determination of Carbon-to-Carbon Double Bonds in the s-PAA Polymers)

The NMR Alkene Content Method is used to determine the percentage, on a molar basis, of alkene terminal moieties per monomer present in a SAP material sample. In this method, proton NMR spectroscopy is used to analyze a sample of SAP material in deuterated water, and peaks corresponding to alkene protons and backbone monomer protons, respectively, are identified, integrated, and ratioed to determine the mole percent alkene terminal moieties per polymer backbone monomer unit (referred to herein as carbon-to-carbon double bonds).

Approximately 0.1 mL of SAP solution is diluted with approximately 1 mL of deuterated water D2O and stirred over at least 5 min to ensure homogeneity. The sample is then transferred to an NMR glass grade tube and placed in the sample holder of a proton NMR instrument. (An example of a suitable instrument is a Bruker NMR device with 400 MHZ field strength. Instruments of other makes and other field strengths, even including “low-field” instruments operating as low as 60 MHZ, can successfully be used to perform this method.) A noesy-presat sequence is used to acquire the data and suppress the residual water signal. One of skill will be familiar with appropriate choice of other specific data collection parameters. Appropriate parameters used with the exemplary 400-MHz Bruker instrument above are: acquisition time (FID length) of 4.1 s, relaxation time of 8 s, 90-degree pulse widths, spectral width of 20 ppm, 64k points in the FID, and 64 repetition scans used. In the Fourier transform step, exponential apodization is used with 0.3-Hz line broadening, and the spectrum is phased into absorption. A spline baseline correction is used to ensure flat baseline on either side of peaks to be integrated.

The peak in the NMR spectrum corresponding to one of the two terminal alkene protons at a chemical shift of approximately 5.35 ppm is identified, if present, and integrated. (To confirm the identify of a ˜5.35 ppm proton peak as terminal alkene proton a standard edited 1H-13C HSQC sequence can be used (following e.g. W. Willker, D. Leibfritz, R. Kerssebaum & W. Bermel, Magn. Reson. Chem. 31, 287-292 (1993)) to determine that the alkene signals seen in the 1D-1H spectrum are both attached to the same methylene (secondary) carbon (—CH2).) If no such peak is present, this reported as no measurable terminal alkene content. Otherwise, the CH backbone signal at a ˜1.8 ppm (arising from the single proton on tertiary carbon of polyacrylic acid each monomer unit) is integrated. The ratio of the area of the ˜5.35-ppm alkene peak to that of the ˜1.8 -ppm CH backbone peak is calculated and is reported as a percentage to the nearest 0.1%.

Gel Permeation Chromatography with Multi-Angle Light Scattering and Refractive Index Detection (GPC-MALS/RI) for Polymer Molecular Weight Distribution Measurement Gel Permeation Chromatography (GPC) with Multi-Angle Light Scattering (MALS) and Refractive Index (RI) Detection (GPC-MALS/RI) permits the measurement of absolute weight average molecular weight Mw of a polymer without the need for column calibration methods or standards. The GPC system allows molecules to be separated as a function of their molecular size. MALS and RI allow information to be obtained on the number average (Mn) and weight average (Mw) molecular weight.

The Mw distribution of water-soluble polymers, such as s-PAA polymers, is typically measured by using a Liquid Chromatography system consisting generally of a pump system, an autosampler (e.g., Agilent 1260 Infinity pump system with OpenLab Chemstation software, Agilent Technology, Santa Clara, CA, USA), and a column set of appropriate dimensions (e.g., Waters ultrahydrogel guard column, 6 mm ID×40 mm length, two ultrahydrogel linear columns, 7.8 mm ID×300 mm length, Waters Corporation of Milford, Mass., USA) which is typically operated at 40° C.

The column set comprises one or typically more subsequently connected columns with varying pore-sizes graded for different molecular weight polymers and columns are generally selected such to provide resolution of wide and relevant molecular weights range.

Commonly, the mobile phase is for example 0.1M sodium nitrate in water containing 0.02% sodium azide and is pumped at a flow rate of about 1 mL/min, isocratically. A multiangle light scattering (MALS) detector (e.g. DAWN®) and a differential refractive index (RI) detector (e.g. Wyatt Technology of Santa Barbara, Calif., USA) controlled by respective software packages, e.g. Wyatt Astra®, are used.

A sample is typically prepared by dissolving polymer materials, such as s-PAA polymers, in the mobile phase at about 1 mg per ml and by mixing the solution for overnight hydration at room temperature. The sample is filtered through a membrane filter (e.g. a 0.8 μm Versapor filter, PALL, Life Sciences, NY, USA) into the LC autosampler vial using a syringe before the GPC analysis.

A dn/dc (differential change of refractive index with concentration) value is typically measured on the polymer materials of interest and used for the number average molecular weight and weight average molecular weight determination by the respective detector software.

### Urine Permeability Measurement (UPM) Test Method

### Lab Conditions:

This test has to be performed in a climate conditioned room at standard conditions of 23° C.±2° C. temperature and 45%±10% relative humidity.

### Urine Permeability Measurement System

This method determined the permeability of a swollen hydrogel layer 1318. The equipment used for this method is described below. This method is closely related to the SFC (Saline Flow Conductivity) test method of the prior art.

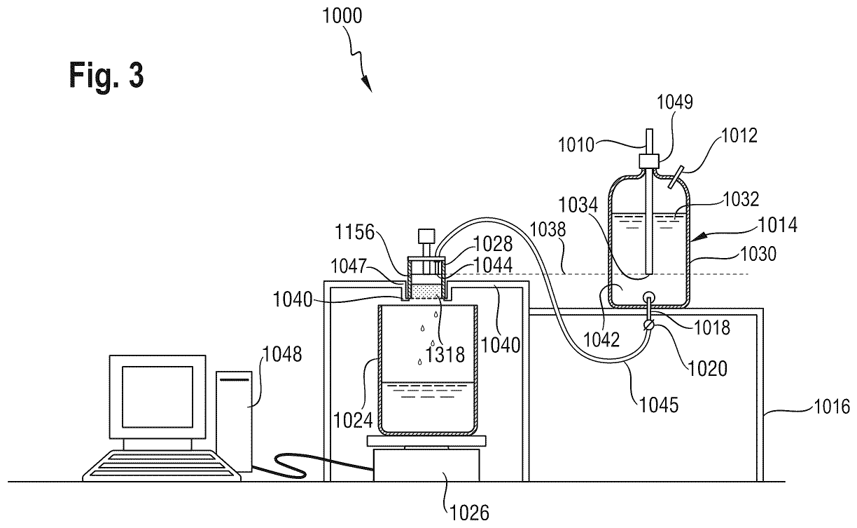

FIG. 3 shows permeability measurement system 1000 set-up with the constant hydrostatic head reservoir 1014, open-ended tube for air admittance 1010, stoppered vent for refilling 1012, laboratory reck 1016, delivery tube 1018 with flexible tube 1045 with Tygon tube nozzle 1044, stopcock 1020, cover plate 1047 and supporting ring 1040, receiving vessel 1024, balance 1026 and piston/cylinder assembly 1028.

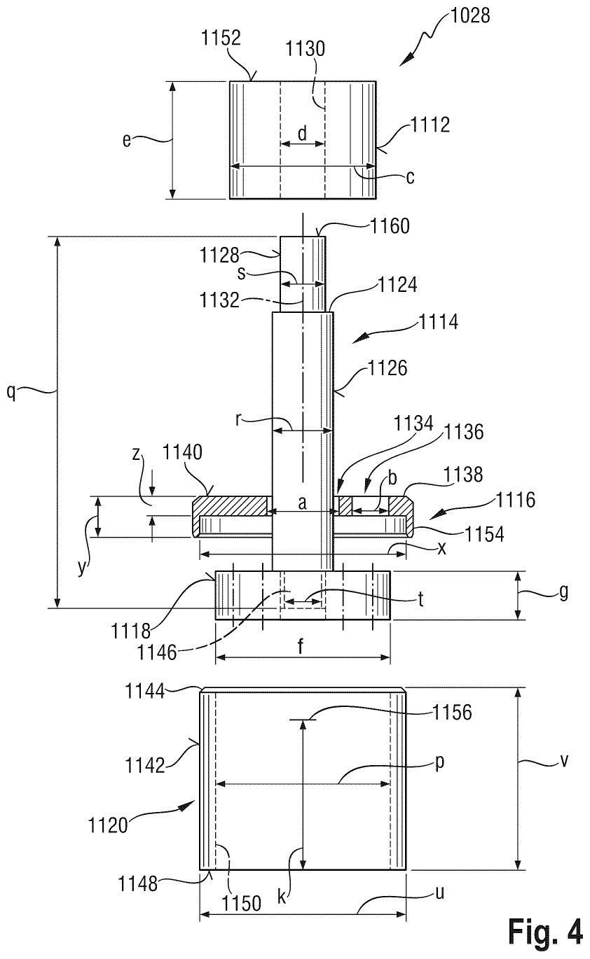

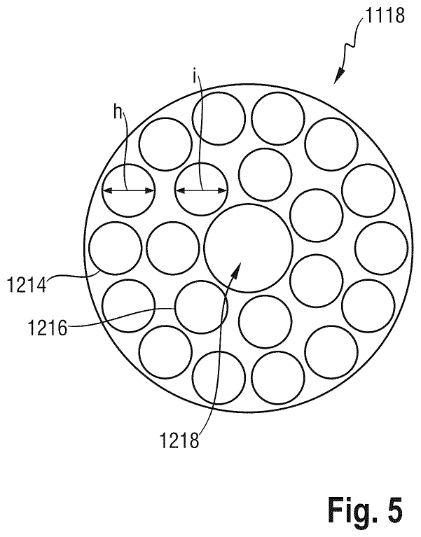

FIG. 4 shows the piston/cylinder assembly 1028 comprising a metal weight 1112, piston shaft 1114, piston head 1118, lid 1116, and cylinder 1120. The cylinder 1120 is made of transparent polycarbonate (e.g., Lexan®) and has an inner diameter p of 6.00 cm (area=28.27 cm2) with inner cylinder walls 1150 which are smooth. The bottom 1148 of the cylinder 1120 is faced with a stainless-steel screen cloth (ISO 9044 Material 1.4401, mesh size 0.038 mm, wire diameter 0.025 mm) (not shown) that is bi-axially stretched to tautness prior to attachment to the bottom 1148 of the cylinder 1120. The piston shaft 1114 is made of transparent polycarbonate (e.g., Lexan®) and has an overall length q of approximately 127 mm. A middle portion 1126 of the piston shaft 1114 has a diameter r of 22.15 (±0.02) mm. An upper portion 1128 of the piston shaft 1114 has a diameter s of 15.8 mm, forming a shoulder 1124. A lower portion 1146 of the piston shaft 1114 has a diameter t of approximately ⅝ inch (15.9 mm) and is threaded to screw firmly into the center hole 1218 (see FIG. 5) of the piston head 1118. The piston head 1118 is perforated, made of transparent polycarbonate (e.g., Lexan®), and is also screened with a stretched stainless-steel screen cloth (ISO 9044 Material 1.4401, mesh size 0.038 mm, wire diameter 0.025 mm) (not shown). The weight 1112 is stainless steel, has a center bore 1130, slides onto the upper portion 1128 of piston shaft 1114 and rests on the shoulder 1124. The combined weight of the piston head 1118, piston shaft 1114 and weight 1112 is 596 g (±6 g), which corresponds to 0.30 psi over the inner area of the cylinder 1120. The combined weight may be adjusted by drilling a blind hole down a central axis 1132 of the piston shaft 1114 to remove material and/or provide a cavity to add weight. The cylinder lid 1116 has a first lid opening 1134 in its center for vertically aligning the piston shaft 1114 and a second lid opening 1136 near the edge 1138 for introducing fluid from the constant hydrostatic head reservoir 1014 into the cylinder 1120.

A first linear index mark (not shown) is scribed radially along the upper surface 1152 of the weight 1112, the first linear index mark being transverse to the central axis 1132 of the piston shaft 1114. A corresponding second linear index mark (not shown) is scribed radially along the top surface 1160 of the piston shaft 1114, the second linear index mark being transverse to the central axis 1132 of the piston shaft 1114. A corresponding third linear index mark (not shown) is scribed along the middle portion 1126 of the piston shaft 1114, the third linear index mark being parallel with the central axis 1132 of the piston shaft 1114. A corresponding fourth linear index mark (not shown) is scribed radially along the upper surface 1140 of the cylinder lid 1116, the fourth linear index mark being transverse to the central axis 1132 of the piston shaft 1114. Further, a corresponding fifth linear index mark (not shown) is scribed along a lip 1154 of the cylinder lid 1116, the fifth linear index mark being parallel with the central axis 1132 of the piston shaft 1114. A corresponding sixth linear index mark (not shown) is scribed along the outer cylinder wall 1142, the sixth linear index mark being parallel with the central axis 1132 of the piston shaft 1114. Alignment of the first, second, third, fourth, fifth, and sixth linear index marks allows for the weight 1112, piston shaft 1114, cylinder lid 1116, and cylinder 1120 to be repositioned with the same orientation relative to one another for each measurement.

The cylinder 1120 specification details are:
Outer diameter u of the Cylinder 1120: 70.35 mm (±0.05 mm)
Inner diameter p of the Cylinder 1120: 60.0 mm (±0.05 mm)
Height v of the Cylinder 1120: 60.5 mm. Cylinder height must not be lower than 55.0 mm!

The cylinder lid 1116 specification details are:
Outer diameter w of cylinder lid 1116: 76.05 mm (±0.05 mm)
Inner diameter x of cylinder lid 1116: 70.5 mm (±0.05 mm)
Thickness y of cylinder lid 1116 including lip 1154: 12.7 mm
Thickness z of cylinder lid 1116 without lip 1154: 6.35 mm
Diameter a of first lid opening 1134: 22.25 mm (±0.02 mm)
Diameter b of second lid opening 1136: 12.7 mm (±0.1 mm)
Distance between centers of first and second lid openings 1134 and 1136:23.5 mm

The weight 1112 specification details are:
Outer diameter c: 50.0 mm
Diameter d of center bore 1130: 16.0 mm
Height e: 39.0 mm

The piston head 1118 specification details are:
Diameter f: 59.7 mm (±0.05 mm)
Height g: 16.5 mm. Piston head height must not be less than 15.0 mm.
Outer holes 1214 (14 total) with a 9.30 (±0.25) mm diameter h, outer holes 1214 equally spaced with centers being 23.9 mm from the center of center hole 1218.
Inner holes 1216 (7 total) with a 9.30 (±0.25) mm diameter i, inner holes 1216 equally spaced with centers being 13.4 mm from the center of center hole 1218.
Center hole 1218 has a diameter j of approximately ⅝ inches (15.9 mm) and is threaded to accept a lower portion 1146 of piston shaft 1114.

Prior to use, the stainless-steel screens (not shown) of the piston head 1118 and cylinder 1120 should be inspected for clogging, holes or over-stretching and replaced when necessary. A urine permeability measurement apparatus with damaged screen can deliver erroneous UPM results and must not be used until the screen has been replaced.

A 5.00 cm mark 1156 is scribed on the cylinder 1120 at a height k of 5.00 cm (±0.05 cm) above the screen (not shown) attached to the bottom 1148 of the cylinder 1120. This marks the fluid level to be maintained during the analysis. Maintenance of correct and constant fluid level (hydrostatic pressure) is critical for measurement accuracy.

A constant hydrostatic head reservoir 1014 is used to deliver salt solution 1032 to the cylinder 1120 and to maintain the level of salt solution 1032 at a height k of 5.00 cm above the screen (not shown) attached to the bottom 1148 of the cylinder 1120. The bottom 1034 of the air-intake tube 1010 is positioned so as to maintain the salt solution 1032 level in the cylinder 1120 at the required 5.00 cm height k during the measurement, i.e., bottom 1034 of the air tube 1010 is in approximately same plane 1038 as the 5.00 cm mark 1156 on the cylinder 1120 as it sits on the cover plate 1047 and supporting ring 1040 (with circular inner opening of not less than 64 mm diameter) above the receiving vessel 1024.

The cover plate 1047 and supporting ring 1040 are parts as used in the equipment used for the method “K(t) Test Method (Dynamic Effective Permeability and Uptake Kinetics Measurement Test method)” as described in EP 2 535 027 A1 and is called “Zeitabhängiger Durchlässigkeitsprüfstand” or “Time Dependent Permeability Tester”, Equipment No. 03-080578 and is commercially available at BRAUN GmbH, Frankfurter Str. 145, 61476 Kronberg, Germany. Upon request, detailed technical drawings are also available.

Proper height alignment of the air-intake tube 1010 and the 5.00 cm mark 1156 on the cylinder 1120 is critical to the analysis. A suitable reservoir 1014 consists of a jar 1030 containing: a horizontally oriented L-shaped delivery tube 1018 connected to a flexible tube 1045 (e.g. Tygon tube, capable to connect nozzle and reservoir outlet) and to a Tygon tube nozzle 1044 (inner diameter at least 6.0 mm, length appr. 5.0 cm) for fluid delivery, a vertically oriented open-ended tube 1010 for admitting air at a fixed height within the constant hydrostatic head reservoir 1014, and a stoppered vent 1012 for re-filling the constant hydrostatic head reservoir 1014. Tube 1010 has an internal diameter of approximately 12 mm, but not less than 10.5 mm. The delivery tube 1018, positioned near the bottom 1042 of the constant hydrostatic head reservoir 1014, contains a stopcock 1020 for starting/stopping the delivery of salt solution 1032. The outlet 1044 of the delivery flexible tube 1045 is dimensioned (e.g. outer diameter 10 mm) to be inserted through the second lid opening 1136 in the cylinder lid 1116, with its end positioned below the surface of the salt solution 1032 in the cylinder 1120 (after the 5.00 cm height of the salt solution 1032 is attained in the cylinder 1120). The air-intake tube 1010 is held in place with an o-ring collar 1049. The constant hydrostatic head reservoir 1014 can be positioned on a laboratory reck 1016 at a suitable height relative to that of the cylinder 1120. The components of the constant hydrostatic head reservoir 1014 are sized so as to rapidly fill the cylinder 1120 to the required height (i.e., hydrostatic head) and maintain this height for the duration of the measurement. The constant hydrostatic head reservoir 1014 must be capable of delivering salt solution 1032 at a flow rate of at least 2.6 g/sec for at least 10 minutes.

The piston/cylinder assembly 1028 is positioned on the supporting ring 1040 in the cover plate 1047 or suitable alternative rigid stand. The salt solution 1032 passing through the piston/cylinder assembly 1028 containing the swollen hydrogel layer 1318 is collected in a receiving vessel 1024, positioned below (but not in contact with) the piston/cylinder assembly 1028.

The receiving vessel 1024 is positioned on the balance 1026 which is accurate to at least 0.001 g. The digital output of the balance 1026 is connected to a computerized data acquisition system 1048.

### Preparation of Reagents (Not Illustrated)

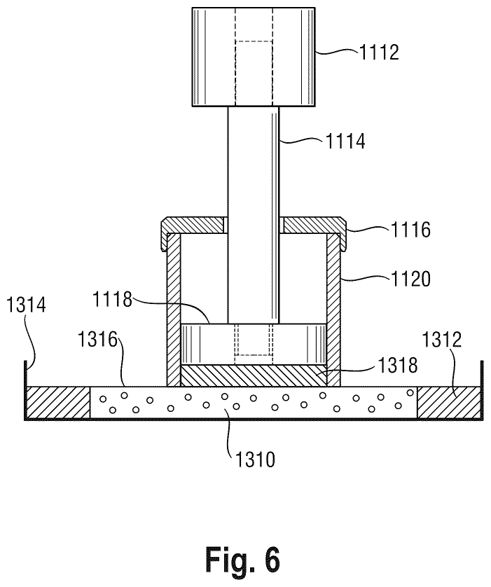

Jayco Synthetic Urine (JSU) 1312 (see FIG. 6) is used for a swelling phase (see UPM Procedure below) and 0.118 M Sodium Chloride (NaCl) Solution 1032 is used for a flow phase (see UPM Procedure below). The following preparations are referred to a standard 1 liter volume. For preparation of volumes other than 1 liter, all quantities are scaled accordingly.

JSU: A 1L volumetric flask is filled with distilled water to 80% of its volume, and a magnetic stir bar is placed in the flask. Separately, using a weighing paper or beaker the following amounts of dry ingredients are weighed to within ±0.01 g using an analytical balance and are added quantitatively to the volumetric flask in the same order as listed below. The solution is stirred on a suitable stir plate until all the solids are dissolved, the stir bar is removed, and the solution diluted to 1L volume with distilled water. A stir bar is again inserted, and the solution stirred on a stirring plate for a few minutes more.

### Quantities of Salts to Make 1 Liter of Jayco Synthetic Urine

Potassium Chloride (KCl) 2.00 g
Sodium Sulfate (Na2SO4) 2.00 g
Ammonium dihydrogen phosphate (NH4H2PO4) 0.85 g
Ammonium phosphate, dibasic ((NH4)2HPO4) 0.15 g
Calcium chloride (CaCl2) 0.19 g—[or hydrated calcium chloride (CaCl2·2H2O) 0.25 g]
Magnesium chloride (MgCl2) 0.23 g—[or hydrated magnesium chloride (MgCl2·6H2O) 0.50 g]

To make the preparation faster, potassium chloride, sodium sulfate, ammonium dihydrogen phosphate, ammonium phosphate (dibasic) and magnesium chloride (or hydrated magnesium chloride) are combined and dissolved in the 80% of distilled water in the 1 L volumetric flask. Calcium chloride (or hydrated calcium chloride) is dissolved separately in approximately 50 ml distilled water (e.g. in a glass beaker) and the calcium chloride solution is transferred to the 1 L volumetric flask after the other salts are completely dissolved therein. Afterwards, distilled water is added to 1 L (1000 ml±0.4 ml) and the solution is stirred for a few minutes more. Jayco synthetic urine may be stored in a clean plastic container for 10 days. The solution should not be used if it becomes cloudy.

0.118 M Sodium Chloride (NaCl) Solution: 0.118 M Sodium Chloride is used as salt solution 1032. Using a weighing paper or beaker 6.90 g (±0.01 g) of sodium chloride is weighed and quantitatively transferred into a 1 L volumetric flask (1000 ml±0.4 ml); and the flask is filled to volume with distilled water. A stir bar is added and the solution is mixed on a stirring plate until all the solids are dissolved.

The conductivity of the prepared Jayco solution must be in the range of appr. 7.48-7.72 mS/cm and of the prepared 0.118 M Sodium Chloride (NaCl) Solution in the range of appr. 12.34-12.66 mS/cm (e.g. measured via COND 70 INSTRUMENT without CELL, #50010522, equipped with Cell VPT51-01 C=0.1 from xs instruments or via LF 320/Set, #300243 equipped with TetraCon 325 from WTW or COND 330i, #02420059 equipped with TetraCon 325 from WTW). The surface tension of each of the solutions must be in the range of 71-75 mN/m (e.g. measured via tensiometer K100 from Kruess with Pt plate).

### Test Preparation

Using a solid reference cylinder weight (not shown) (50 mm diameter; 128 mm height), a caliper gauge (not shown) (measurement range 25 mm, accurate to 0.01 mm, piston pressure max. 50 g; e.g. Mitutoyo Digimatic Height Gage) is set to read zero. This operation is conveniently performed on a smooth and level bench (not shown) of at least approximately 11.5 cm×15 cm. The piston/cylinder assembly 1028 without superabsorbent polymer particles is positioned under the caliper gauge (not shown) and a reading, L1, is recorded to the nearest 0.01 mm.

The constant hydrostatic head reservoir 1014 is filled with salt solution 1032. The bottom 1034 of the air-intake tube 1010 is positioned so as to maintain the top part (not shown) of the liquid meniscus (not shown) in the cylinder 1120 at the 5.00 cm mark 1156 during the measurement. Proper height alignment of the air-intake tube 1010 at the 5.00 cm mark 1156 on the cylinder 1120 is critical to the analysis.

The receiving vessel 1024 is placed on the balance 1026 and the digital output of the balance 1026 is connected to a computerized data acquisition system 1048. The cover plate 1047 with the supporting ring 1040 is positioned above the receiving vessel 1024.

### UPM Procedure

1.5 g (±0.05 g) of superabsorbent polymer particles is weighed onto a suitable weighing paper or weighing aid using an analytical balance. The moisture content of the superabsorbent polymer particles is measured according to the EDANA Moisture Content Test Method NWSP 230.0.R2 (15) or via a Moisture Analyzer (HX204 from Mettler Toledo, drying temperature 130° C., starting superabsorbent polymer particles weight 3.0 g (±0.5 g), stop criterion 1 mg/140 s). If the moisture content of the superabsorbent polymer particles is greater than 3wt %, then the superabsorbent polymer particles are dried to a moisture level of <3wt %, e.g. in an oven at 105° C. for 3 h or e.g. at 120° C. for 2 h. Agglomerated superabsorbent polymer particles are dried if moisture level is greater than 5 wt %, e.g. in an oven at 105° C. for 3 h or e.g. at 120° C. for 2 h.

The empty cylinder 1120 is placed on a level benchtop 1046 (not shown) and the superabsorbent polymer particles are quantitatively transferred into the cylinder 1120. The superabsorbent polymer particles are evenly dispersed on the screen (not shown) attached to the bottom 1148 of the cylinder 1120 while rotating the cylinder 1120, e.g. aided by a (manual or electrical) turn table (e.g., petriturn-E or petriturn-M from Schuett). It is important to have an even distribution of particles on the screen (not shown) attached to the bottom 1148 of the cylinder 1120 to obtain the highest precision result. After the superabsorbent polymer particles have been evenly distributed on the screen (not shown) attached to the bottom 1148 of the cylinder 1120 particles must not adhere to the inner cylinder walls 1150. The piston shaft 1114 is inserted through the first lid opening 1134, with the lip 1154 of the lid 1116 facing towards the piston head 1118. The piston head 1118 is carefully inserted into the cylinder 1120 to a depth of a few centimeters. The lid 1116 is then placed onto the upper rim 1144 of the cylinder 1120 while taking care to keep the piston head 1118 away from the superabsorbent polymer particles. The weight 1112 is positioned on the upper portion 1128 of the piston shaft 1114 so that it rests on the shoulder 1124 such that the first and second linear index marks are aligned. The lid 1116 and piston shaft 1126 are then carefully rotated so as to align the third, fourth, fifth, and sixth linear index marks are then aligned with the first and the second linear index marks. The piston head 1118 (via the piston shaft 1114) is then gently lowered to rest on the dry superabsorbent polymer particles. Proper seating of the lid 1116 prevents binding and assures an even distribution of the weight on the hydrogel layer 1318.

### Swelling Phase

A fritted disc of at least 8 cm diameter (e.g. 8-9 cm diameter) and at least 5.0 mm thickness (e.g. 5-7 mm thickness) with porosity “coarse” or “extra coarse” (e.g. Chemglass Inc. #CG 201-51, coarse porosity; or e.g. Robu 1680 with porosity 0) 1310 is placed in a wide flat-bottomed Petri dish 1314 and JSU 1312 is added by pouring JSU 1312 onto the center of the fritted disc 1310 until JSU 1312 reaches the top surface 1316 of the fritted disc 1310. The JSU height must not exceed the height of the fritted disc 1310. It is important to avoid any air or gas bubbles entrapped in or underneath the fritted disc 1310.

The entire piston/cylinder assembly 1028 is lifted and placed on the fritted disc 1310 in the Petri dish 1314. JSU 1312 from the Petri dish 1314 passes through the fritted disc 1310 and is absorbed by the superabsorbent polymer particles (not shown) to form a hydrogel layer 1318. The JSU 1312 available in the Petri dish 1314 should be enough for all the swelling phase. If needed, more JSU 1312 may be added to the Petri dish 1314 during the hydration period to keep the JSU 1312 level at the top surface 1316 of the fritted disc 1310. After a period of 60 minutes, the piston/cylinder assembly 1028 is removed from the fritted disc 1310, taking care to ensure the hydrogel layer 1318 does not lose JSU 1312 or take in air during this procedure. The piston/cylinder assembly 1028 is placed under the caliper gauge (not shown) and a reading, L2, is recorded to the nearest 0.01 mm. If the reading changes with time, only the initial value is recorded. The thickness of the hydrogel layer 1318, L0 is determined from L2-L1 to the nearest 0.1 mm.

The piston/cylinder assembly 1028 is transferred to the supporting ring 1040 in the cover plate 1047. The constant hydrostatic head reservoir 1014 is positioned such that the delivery tube nozzle 1044 is placed through the second lid opening 1136. The measurement is initiated in the following sequence:
a) The stopcock 1020 of the constant hydrostatic head reservoir 1014 is opened to permit the salt solution 1032 to reach the 5.00 cm mark 1156 on the cylinder 1120. This salt solution 1032 level should be obtained within 10 seconds of opening the stopcock 1020.
b) Once 5.00 cm of salt solution 1032 is attained, the data collection program is initiated.

With the aid of a computer 1048 attached to the balance 1026, the quantity g (in g to accuracy of 0.001 g) of salt solution 1032 passing through the hydrogel layer 1318 is recorded at intervals of 20 seconds for a time period of 10 minutes. At the end of 10 minutes, the stopcock 1020 on the constant hydrostatic head reservoir 1014 is closed.

The data from 60 seconds to the end of the experiment are used in the UPM calculation. The data collected prior to 60 seconds are not included in the calculation.

For each time period of 20 seconds (time t(i−1) to ti) after the initial 60 seconds of the experiment, the respective flow rate Fs(t) (in g/s) and the respective mid-point of the time t(½)t (in s) is calculated according to the following formulas:

$${Fs}_{( t )} = \frac{( {g}_{( i - 1 )} - {g}_{( i )} )}{( {t}_{( i - 1 )} - {t}_{( i )} )} and {t}_{{( 1 / 2 )}_{t}} = \frac{( {t}_{( i - 1 )} + {t}_{( i )} )}{2} \qquad ( XII )$$

The flow rate Fs(t) of each time interval (t(i−1) to ti) is plotted versus the mid-point of the time t(½) t of the time interval (t(i−1) to ti). The intercept is calculated as Fs(t=0).

### Calculation of the Intercept

The intercept is calculated via a best-fit regression line, e.g. as following: the equation for the intercept of the regression line, a, is:

$$a = {y}_{AVG} - b \cdot {x}_{AVG} \qquad ( XIII )$$

where the slope, b, is calculated as:

$$b = \frac{\sum ( x - {x}_{AVG} ) \cdot ( y - {y}_{AVG} )}{\sum {( x - {x}_{AVG} )}^{2}} \qquad ( XIV )$$

and where xAVG and yAVG are the sample means AVERAGE of the known_x's and AVERAGE of the known_y's, respectively.

### Calculation of Urine Permeability Measurement Q

The intercept Fs(t=0) is used to calculate Q according to the following formula:

$$Q = \frac{{F}_{s} ( t = 0 ) \cdot {L}_{0}}{ρ \cdot A \cdot Δ P} \qquad ( XV )$$

where the flow rate Fs(t−0) is given in g/s, L0 is the initial thickness of the hydrogel layer 1318 in cm, ρ is the density of the salt solution 1032 in g/cm3 (e.g. 1.003 g/cm³ at room temperature). A (from the equation above) is the area of the hydrogel layer 1318 in cm2 (e.g. 28.27 cm2), ΔP is the hydrostatic pressure in dyne/cm2 (e.g. 4920 dyne/cm²), and the Urine Permeability Measurement, Q, is in units of cm3sec/g. The average of three determinations should be reported.

| Variable | Description | Unit |
|---|---|---|
| gi | Mass of salt solution 1032 flown through | g |
| | the swollen gel layer (recorded by the | |
| | balance) at the time ti (accuracy 0.001 g) | |
| ti | Time point (every 20 s) | s |
| t(1/2)t | Mid-point of time for the respective time | s |
| | interval ti-1 to ti | |
| Fst | Flow Rate at the time interval ti-1 to ti | g/s |
| Fs (t = 0) | Intercept flow rate at t = 0 s from the plot | g/s |
| | of the flow rate Fs(t) vs. the | |
| | mid-point of time t(1/2)t. | |
| L0 | Thickness of the swollen gel layer | cm |
| | (swollen with JSU 1312) before the salt | |
| | solution 1032 flows through the gel layer. | |
| ρ | Density of the salt solution 1032 | g/cm3 |
| | (1.003 g/cm3) | |
| A | Area of the swollen gel layer (28.27 cm2) | cm2 |
| ΔP | Hydrostatic pressure across the gel layer | dyne/cm2 |
| | (4920 dyne/cm2) | |
| Q | Urine Permeability Measurement | cm3 * sec/g |

Capacity is determined according to the Centrifuge Retention Capacity (CRC) test method as set out in EDANA NWSP 241.0.R2(15). In deviation from EDANA NWSP 241.0.R2(15), CRC measurement is started at a lower end of 24.2 g/g (instead of 27.19 g/g as set out in EDANA NWSP 241.0.R2(15).

The Absorption Against Pressure (AAP) test method is set out in EDANA method NWSP 242.0.R2 (15). In deviation from the EDANA method, a pressure of 0.7 psi is applied (instead of a pressure of 0.3 psi provided in EDANA method NWSP 242.0.R2 (15)).

Amount of extractables is measured in accordance with EDANA test method NWSP 270.0.R2 (15). The following deviations from EDANA test method NWSP 270.0.R2 (15) apply herein:
9. Procedure (procedural steps not described below are carried out without deviation from EDANA test method NWSP 270.0.R2 (15):
9.2 Accurately add 200.0±0.1 ml of saline solution to dispenser with a volume of 200 ml (instead of a 250 ml beaker or conical flask as is set out in EDANA test method NWSP 270.0.R2 (15)).
9.4 Add the 0.95 to 1.05 g of a SAP particle sample by weighing it directly into the 250 ml Erlenmeyer flask and add the magnet coin (instead of adding the sample to the weighing vessel or laboratory paper and tare the balance again, as is set out in EDANA test method NWSP 270.0.R2 (15)). Fill the saline solution to the Erlenmeyer flask only at the start of the extraction time.
9.7 Stopper/Cover/Seal the beaker or conical flask, and stir the solution at a rate of 250±50 r.min-1 for 16 hours (instead of for 1 hour, as is set out in EDANA test method NWSP 270.0.R2 (15)).
9.8 Prepare a titration blank by treating 200.0±0.1 ml of the same batch of saline solution as used for the sample preparation in the same way. Deviation from EDANA test method NWSP 270.0.R2 (15): n=2.
9.9 Stop stirring the solutions, and filter the extracted sample directly with a screen-covered beaker (CCRC beaker) with no downtime (instead of allowing the gel to completely settle to the bottom of the beaker, as is set out in EDANA test method NWSP 270.0.R2 (15)).

### EXAMPLES

Various examples of the present invention as well as comparative examples have been prepared and evaluated.

The inventive examples differ
a) in the mol percent of carbon-to-carbon double bonds comprised in the s-PAA polymers used in making the SAP particles,
b) in the weight average molecular weight Mw of the s-PAA polymers,
c) in the amount (in weight-%) of s-PAA polymers used in making the SAP particles, ranging from 5 weight-% to 66.7 weight-%,
d) in the source of the s-PAA polymers (i.e. s-PAA polymers obtained from different SAP particle degradation methods), and
e) in the amount of cross-linkers added in the method of making the SAP particles, including two examples (A8 and A9) for which no cross-linker was provided at all (i.e. facilitating cross-linking solely by the s-PAA polymers having carbon-to-carbon double bonds).

In the making of the comparative examples either no s-PAA polymers were used at all (Comparative Examples C1,C2 and C7), or commercially available s-PAA polymers were used (Comparative Examples C3 to C6), for which no carbon-to-carbon double bonds can be detected. This finding confirmed the understanding that commercially available s-PAA polymers do not comprise any carbon-to-carbon double bonds.

TABLE 1
Overview of Examples A1 to 11 and Comparative Examples C1 to C7

| | | | | | | Terminal | |
|---|---|---|---|---|---|---|---|
| | | | | Crosslinker: | | Alkenyl | solid |
| | | | Mw of | Mol. Ratio | | groups (1H- | content |
| | | | s-PAA | of PEG 700- | s-PAA | NMR) | “w” at |
| | Base | s-PAA | polymer, | DA vs AA | add-on | % mol in s- | synthesis, |
| Example | Polymer | polymer | kDa | monomer | level, % wt | PAA | % wt |
| C1 | BP C1/7 | None | — | 0.075 | 0 | — | 28.0 |
| C2 | BP C2 | None | — | 0.18 | 0 | — | 28.2 |
| C3 | BP C3 | Sigma-Aldrich | 100 *⁾ | 0.075 | 5 | 0 | 28.0 |
| C4 | BP C4 | Sigma-Aldrich | 100 *⁾ | 0.075 | 10 | 0 | 27.9 |
| C5 | BP C5 | Sokalan PA | 223 | 0.075 | 5 | 0 | 28.0 |
| | | 110 S (BASF) | | | | | |
| C6 | BP C6 | Sokalan PA | 223 | 0.075 | 10 | 0 | 31.8 |
| | | 110 S (BASF) | | | | | |
| C7 | BP C1/7 | None | — | 0.075 | 0 | — | 28.0 |
| A1 | BP A1 | PAA A1 | 134 | 0.075 | 5 | n.a. | 28.6 |
| A2 | BP A2 | PAA A2 | 277 | 0.075 | 5 | 0.22 | 27.5 |
| A3 | BP A3 | PAA A3 | 525 | 0.075 | 10 | n.a. | 27.0 |
| A4 | BP A4 | PAA A456 | 285 | 0.075 | 10 | 0.22 | 25.1 |
| A5 | BP A5 | | 285 | 0.04 | 30 | 0.22 | 25.2 |
| A6 | BP A6 | | 285 | 0.075 | 15 | 0.22 | 26.6 |
| A7 | BP A7 | PAA A7 | 285 | 0.04 | 20 | 0.26 | 33.9 |
| A8 | BP A8 | PAA A8 | 239 | 0 | 20 | 0.3 | 38.9 |
| A9 | BP A9 | PAA A9 | 229 | 0 | 66.7 | 0.24 | 23.0 |
| A10 | BP A10 | PAA A10 | 1080 | 0.075 | 5 | 0.1 | 28.0 |
| A11 | BP A11 | PAA A11 | 418 | 0.075 | 5 | 0.04 | 28.0 |

*⁾ the molecular weight of the s-PAA polymer of comparative example C3 and C4 was not determined by the test method set out herein. Instead, the molecular weight was given on the label of the commercial s-PAA polymer was taken.

### Preparation of Base Polymer BP C1/7 of Comparative Examples C1 and C7

A 20,000 ml resin kettle (equipped with a four-necked glass cover closed with septa, suited for the introduction of a thermometer, syringe needles) was charged with about 5097.0 g of ice (ca. 50% of the total amount of ice: 9676.1 g ice prepared from deionized water). A magnetic stirrer, capable of mixing the whole content (when liquid), was added and stirring was started.

About 200.0 g of deionized water was taken to dissolve 5.181 g of “KPS” (=potassium peroxydisulfate, from Sigma Aldrich) e.g. in a glass beaker of 250 mL volume. The vessel with the “KPS” solution was closed and set aside.

About 10.0 g of deionized water was taken to dissolve 0.112 g of “ASC” (=Ascorbic Acid, from Sigma Aldrich) e.g. in a glass vial of 40 mL volume. The vessel with the “ASC” solution was closed with a plastic snap-on cap and set aside.

200.0 g of deionized water was taken to dissolve 33.589 g of “PEG700-DA” (=polyethylene glycol diacrylate of Mn˜700 Da from Sigma Aldrich) e.g. in a glass beaker. The beaker with the “PEG700-DA” solution was covered e.g. with parafilm and set aside.

The full amount of 4600.3 g of glacial AA (=acrylic acid) was added to the ice in the resin kettle while stirring was continued.

A thermometer was introduced and in total 3472.6 g of 50% w NaOH (sodium hydroxide) solution (for analysis, from Merck KGaA) and the remaining amount of ice (prepared from de-ionized water) were added subsequently in portions such that the temperature was below 30° C.

The “PEG700-DA” solution was added to the mixture of AA, NaOH solution and ice at a temperature below 30° C. while stirring was continued. The beaker that contained the “PEG700-DA” solution was washed 2×with deionized water in an amount of about 10% of the “PEG700-DA” solution volume per wash. The wash water of both washing steps was added to the stirred mixture.

Deionized water (the remaining amount required to achieve the total amount of (ice+water) of 11888.3 g was added to the stirred mixture.

Then, the resin kettle was closed, and a pressure relief was provided e.g. by puncturing two syringe needles through the septa. The solution was then purged vigorously with argon via an 80 cm injection needle at about 0.4 bar while stirring at about 400-600 RPM. The argon stream was placed close to the stirrer for efficient and fast removal of dissolved oxygen.

After about 1 hour of Argon purging and stirring, the “ASC” solution was added to the reaction mixture at a temperature of about 20° C. via plastic funnel inserted temporarily in one of the resin kettle cover necks while stirring and Argon purging was continued. Thereafter, about 0.022 g of 1% w aqueous solution of hydrogen peroxide H2O2 (Sigma-Aldrich) was added via 1 mL plastic pipette to the “KPS” solution, and the latter was then also added to the reaction mixture via plastic funnel inserted temporarily in one of the resin kettle cover necks while stirring and Argon purging was continued.

After the initiator solutions “KPS” and “ASC” were mixed with the reaction mixture, stirring and Argon purging was continued but the Argon needle was pulled a few cm above the liquid. Within 5 min of “KPS” solution addition, the solution characteristically starts to become turbid or a sudden increase in viscosity was observed. A “gel point” was observed and recorded when the stirbar was not able to rotate freely at the bottom of the resin kettle and the stirring was therefore stopped. Purging with argon was continued at a reduced flow rate (0.2 bar).

The temperature was monitored; typically, it rises from about 20° C. to about 80° C. within 60 minutes. Once the temperature starts to drop from a maximal value, the resin kettle was transferred into a circulation oven (e.g. Binder FED 720 from Binder GmbH) and kept at about 60° C. for about 18 hours.

After this time, the oven was switched off and the resin kettle was allowed to cool down for about 2 hours while remaining in the oven. After that, the gel was removed and broken manually or cut with scissors into smaller pieces. The gel was ground with a grinder (X70G from Scharfen Slicing Machines GmbH with Unger R70 plate system: 3 pre-cutter kidney plates with straight holes at 17 mm diameter), put onto perforated stainless steel dishes (hole diameter 4.8 mm, 50 cm×50 cm, 0.55 mm caliper, 50% open area, from RS; max. height of gel before drying: about 3 cm) and transferred into a circulation oven (Binder FED 720 from Binder GmbH) at about 120° C. for about 20 hours.

The residual moisture content of the dried gel was about 3% by weight (see UPM test method for description of how to determine moisture content).

The dried gel was then ground using a centrifuge mill (Retsch ZM 200 from Retsch GmbH with vibratory feeder DR 100 (setting 50-60), interchangeable sieve with 1.5 mm opening settings, rotary speed 8000 rpm). The milled polymer was then sieved via a sieving machine (AS 400 control from Retsch with sieves DIN/ISO 3310-1 at about 250 rpm for about for 10 min) to the following particle size cuts with the following yields:

| | fines | Collected fraction | crude |
|---|---|---|---|
| sieved fractions | <150 μm | 150-710 μm | >710 μm |
| yield | | 4500 g | |

The fractions “fines” and “crude” have been discarded and not used further.

### Preparation of Base Polymer BP C2 of Comparative Example C2

A 20,000 ml resin kettle (equipped with a four-necked glass cover closed with septa, suited for the introduction of a thermometer, syringe needles) was charged with about 4528.9 g of ice (ca. 50% of the total amount of ice: 8941.1 g ice prepared from deionized water). A magnetic stirrer, capable of mixing the whole content (when liquid), was added and stirring was started.

About 200.0 g of deionized water was taken to dissolve 5.177 g of “KPS” (=potassium peroxydisulfate, from Sigma Aldrich) e.g. in a glass beaker of 250 mL volume. The vessel with the “KPS” solution was closed and set aside.

About 10.0 g of deionized water was taken to dissolve 1.124 g of “ASC” (=Ascorbic Acid, from Sigma Aldrich) e.g. in a glass vial of 40 mL volume. The vessel with the “ASC” solution was closed with a plastic snap-on cap and set aside.

200.0 g of deionized water was taken to dissolve 80.44 g of “PEG700-DA” (=polyethylene glycol diacrylate of Mn˜700 Da from Sigma Aldrich) e.g. in a glass beaker. The beaker with the “PEG700-DA” solution was covered e.g. with parafilm and set aside.

The full amount of 4600.0 g of glacial AA (=acrylic acid) was added to the ice in the resin kettle while stirring was continued.

A thermometer was introduced and in total 3472.7 g of 50% w NaOH (sodium hydroxide) solution (for analysis, from Merck KGaA) and the remaining amount of ice (prepared from de-ionized water) were added subsequently in portions such that the temperature is below 30° C.

The “PEG700-DA” solution was added to the mixture of AA, NaOH solution and ice at a temperature below 30° C. while stirring is continued. The beaker that contained the “PEG700-DA” solution was washed 2× with deionized water in an amount of about 10% of the “PEG700-DA” solution volume per wash. The wash water of both washing steps was added to the stirred mixture.

The remaining amount of deionized water required to achieve the total amount of (ice+water) of 11838.6 g was added to the stirred mixture.

Then, the resin kettle was closed, and a pressure relief was provided e.g. by puncturing two syringe needles through the septa. The solution was then purged vigorously with argon via an 80 cm injection needle at about 0.4 bar while stirring at about 400. The argon stream was placed close to the stirrer for efficient and fast removal of dissolved oxygen.

After about 1 hour of Argon purging and stirring, the “ASC” solution was added to the reaction mixture at a temperature of about 20° C. via plastic funnel inserted temporarily in one of the resin kettle cover necks while stirring and Argon purging was continued. Thereafter, about 0.25 g of 1% w aqueous solution of hydrogen peroxide H2O2 (Sigma-Aldrich) was added via 1 mL plastic pipette to the “KPS” solution, and the latter was then also added to the reaction mixture via plastic funnel inserted temporarily in one of the resin kettle cover necks while stirring and Argon purging was continued.

After the initiator solutions “KPS” and “ASC” were mixed with the reaction mixture, stirring and Argon purging was continued but the Argon needle was pulled a few cm above the liquid. Typically, within 2 min of “KPS” solution addition, the solution characteristically starts to become turbid or a sudden increase in viscosity was observed, typically at temperatures about room temperature. A “gel point” was observed and recorded when the stirbar was not able to rotate freely at the bottom of the resin kettle and the stirring was therefore stopped. Purging with argon was continued at a reduced flow rate (0.2 bar).

The temperature was monitored; typically, it rises from about 20° C. to about 80° C. within 60 minutes. Once the temperature starts to drop from a maximal value, the resin kettle was transferred into a circulation oven (e.g. Binder FED 720 from Binder GmbH) and kept at about 60° C. for about 18 hours.

After this time, the oven was switched off and the resin kettle was allowed to cool down for about 2 hours while remaining in the oven. After that, the gel was removed and broken manually or cut with scissors into smaller pieces. The gel was grinded with a grinder (X70G from Scharfen Slicing Machines GmbH with Unger R70 plate system: 3 pre-cutter kidney plates with straight holes at 17 mm diameter), put onto perforated stainless steel dishes (hole diameter 4.8 mm, 50 cm×50 cm, 0.55 mm caliper, 50% open area, from RS; max. height of gel before drying: about 3 cm) and transferred into a circulation oven (Binder FED 720 from Binder GmbH) at about 120° C. for about 20 hours.

The residual moisture content of the dried gel was about 3% by weight (see UPM test method for description of how to determine moisture content).

The dried gel was then ground using a centrifuge mill (Retsch ZM 200 from Retsch GmbH with vibratory feeder DR 100 (setting 50-60), interchangeable sieve with 1.5 mm opening settings, rotary speed 8000 rpm). The milled polymer was then sieved via a sieving machine (AS 400 control from Retsch with sieves DIN/ISO 3310-1 at about 250 rpm for about for 5-10 min) to the following particle size cuts with the following yields:

| | fines | Collected fraction | crude |
|---|---|---|---|
| sieved fraction | <150 μm | 150-710 μm | >710 μm |
| yield | | ~4200 g | |

The fractions “fines” and “crude” have been discarded and not used further.

### Preparation of Base Polymer BP C3 of Comparative Example C3

A 2,000 ml resin kettle (equipped with a four-necked glass cover closed with septa, suited for the introduction of a thermometer, syringe needles) was placed into an ice bath filled with about 1 liter of water, 100 g of sodium chloride and about 200 g of ice such that the mixture covers about half the height of the resin kettle. The resin kettle was charged with about 80.0 g of solution comprising aqueous polyacrylic acid (s-PAA polymer) of about 35% w concentration wherein the weight average molecular weight Mw reported by the supplier Sigma Aldrich is 100,000 Da. About 591.4 g water was added as ice prepared from DI water and DI water of weight about 443.6 g was also added to the mixture. A magnetic stirrer, capable of mixing the whole content, was added and stirring was started.

As the PAA was fully dispersed, the full amount of 432.5 g of glacial AA (=acrylic acid) was added to the PAA solution in the resin kettle while stirring was continued.

About 20.0 g of deionized water was taken to dissolve 0.4870 g of “KPS” (=potassium peroxydisulfate, from Sigma Aldrich) e.g. in a glass vial of 40 mL volume. The vessel with the “KPS” solution was closed with a plastic snap-on cap and set aside.

About 10.0 g of deionized water was taken to dissolve 0.053 g of “ASC” (=Ascorbic Acid, from Sigma Aldrich) e.g. in a glass vial of 40 mL volume. The vessel with the “ASC” solution was closed with a plastic snap-on cap and set aside.

About 70 g of deionized water was taken to dissolve 3.15 g of “PEG700-DA” (=polyethylene glycol diacrylate of Mn˜700 Da from Sigma Aldrich) e.g. in a 100 mL glass beaker. The beaker with the “PEG700-DA” solution was covered e.g. with parafilm and set aside.

The remaining water up to a final weight of 1136.3 g was added to the resin kettle and stirring was continued as a homogeneous solution was obtained within 1-5 minutes.

A thermometer was introduced and in total 347.5 g of 50% w NaOH (sodium hydroxide) solution (for analysis, from Merck KGaA) were added subsequently in portions such that the temperature was below 30° C.

The “PEG700-DA” solution was added to the mixture of PAA, AA and NaOH solution at a temperature below 30° C. while stirring was continued.

Then, the resin kettle was closed, the ice bath underneath removed, and a pressure relief was provided e.g. by puncturing two syringe needles through the septa. The solution was then purged vigorously with argon via an 80 cm injection needle at about 0.4 bar while stirring at about 400 rpm. The argon stream was placed close to the stirrer for efficient and fast removal of dissolved oxygen.

After about 1 hour of Argon purging and stirring, about 0.026 g (about 1-2 droplets) of 1% w aqueous solution of hydrogen peroxide H2O2 (Sigma-Aldrich) was added via 1 mL plastic pipette to the “KPS” solution, and the latter was then added to the reaction mixture via plastic funnel inserted temporarily in one of the resin kettle cover necks while stirring and Argon purging was continued. Thereafter the “ASC” solution was added to the reaction mixture at a temperature of about 20° C. via plastic funnel inserted temporarily in one of the resin kettle cover necks while stirring and Argon purging was continued.

After the initiator solutions “KPS” and “ASC” were mixed with the reaction mixture, stirring and Argon purging was continued but the Argon needle was pulled a few cm above the liquid. Typically, within 3 min of “ASC” solution addition, the solution characteristically starts to become turbid or a sudden increase in viscosity was observed, typically at temperatures about room temperature. A “gel point” was observed and recorded when the stirbar was not able to rotate freely at the bottom of the resin kettle and the stirring was therefore stopped. Purging with argon was continued at a reduced flow rate (0.2 bar).

The temperature was monitored; typically, it rises from about 20° C. to about 70° C. within 60 minutes. Once the temperature starts to drop from a maximal value, the resin kettle was transferred into a circulation oven (e.g. Binder FED 720 from Binder GmbH) and kept at about 60° C. for about 18 hours.

After this time, the oven was switched off and the resin kettle was allowed to cool down for about 2 hours while remaining in the oven. After that, the gel was removed and broken manually or cut with scissors into smaller pieces. The gel was grinded with a grinder (X70G from Scharfen Slicing Machines GmbH with Unger R70 plate system: 3 pre-cutter kidney plates with straight holes at 17 mm diameter), put onto perforated stainless steel dishes (hole diameter 4.8 mm, 50 cm×50 cm, 0.55 mm caliper, 50% open area, from RS; max. height of gel before drying: about 3 cm) and transferred into a circulation oven (Binder FED 720 from Binder GmbH) at about 120° C. for about 20 hours.

The residual moisture content of the dried gel was below about 3% by weight (see UPM test method for description of how to determine moisture content).

The dried gel was then ground using a centrifuge mill (Retsch ZM 200 from Retsch GmbH with vibratory feeder DR 100 (setting 50-60), interchangeable sieve with 1.5 mm opening settings, rotary speed 8000 rpm). The milled polymer was then sieved via a sieving machine (AS 400 control from Retsch with sieves DIN/ISO 3310-1 at about 250 rpm for about for 5-10 min) to the following particle size cuts with the following yields:

| | fines | Collected fraction | crude |
|---|---|---|---|
| sieved fraction | <150 μm | 150-710 μm | >710 μm |
| yield | | ~350 g | |

The fractions “fines” and “crude” have been discarded and not used further.

### Preparation of Base Polymer BP C4 of Comparative Example C4

A 10,000 ml resin kettle (equipped with a four-necked glass cover closed with septa, suited for the introduction of a thermometer, syringe needles) was charged with about 2392.1 g of ice (ca. 60% of the total amount of ice: 3622.5 g ice prepared from deionized water). A magnetic stirrer, capable of mixing the whole content (when liquid), was added and stirring was started.

About 100.0 g of deionized water was taken to dissolve 2.296 g of “KPS” (=potassium peroxydisulfate, from Sigma Aldrich) e.g. in a glass beaker of 250 mL volume. The vessel with the “KPS” solution was closed and set aside.

About 10.0 g of deionized water was taken to dissolve 0.492 g of “ASC” (=Ascorbic Acid, from Sigma Aldrich) e.g. in a glass vial of 40 mL volume. The vessel with the “ASC” solution was closed with a plastic snap-on cap and set aside.

200.0 g of deionized water was taken to dissolve 14.71 g of “PEG700-DA” (=polyethylene glycol diacrylate of Mn˜700 Da from Sigma Aldrich) e.g. in a glass beaker. The beaker with the “PEG700-DA” solution was covered e.g. with parafilm and set aside.

The full amount of 2020.3 g of glacial AA (=acrylic acid) was added to the ice in the resin kettle while stirring was continued.

An amount of 798.5 g of solution (Sigma Aldrich) comprising aqueous polyacrylic acid of about 35% w concentration wherein the weight average molecular weight Mw as reported by the supplier Sigma Aldrich is 100,000 Da, was added to the mixture in the resin kettle while stirring was continued.

A thermometer was introduced and in total 1735.5 g of 50% w NaOH (sodium hydroxide) solution (for analysis, from Merck KGaA) and the remaining amount of ice (prepared from de-ionized water) were added subsequently in portions such that the temperature was below 30° C.

The “PEG700-DA” solution was added to the mixture of AA, NaOH solution and ice at a temperature below 30° C. while stirring was continued. The beaker that contained the “PEG700-DA” solution was washed 2× with deionized water in an amount of about 10% of the “PEG700-DA” solution volume per wash. The wash water of both washing steps was added to the stirred mixture.

Deionized water (the remaining amount required to achieve the total amount of (ice+water) of 5429.5 g was added to the stirred mixture.

Then, the resin kettle was closed, and a pressure relief was provided e.g. by puncturing two syringe needles through the septa. The solution was then purged vigorously with argon via an 80 cm injection needle at about 0.4 bar while stirring at about 400 rpm. The argon stream was placed close to the stirrer for efficient and fast removal of dissolved oxygen.

After about 1 hour of Argon purging and stirring, the “ASC” solution was added to the reaction mixture at a temperature of about 20° C. via plastic funnel inserted temporarily in one of the resin kettle cover necks while stirring and Argon purging was continued. Thereafter, about 0.99 g of 1% w aqueous solution of hydrogen peroxide H2O2 (Sigma-Aldrich) was added via 1 mL plastic pipette to the “KPS” solution, and the latter was then also added to the reaction mixture via plastic funnel inserted temporarily in one of the resin kettle cover necks while stirring and Argon purging was continued.

After the initiator solutions “KPS” and “ASC” were mixed with the reaction mixture, stirring and Argon purging was continued but the Argon needle was pulled a few cm above the liquid. Typically, within 2 min of “KPS” solution addition, the solution characteristically starts to become turbid or a sudden increase in viscosity was observed, typically at temperatures about room temperature. A “gel point” was observed and recorded when the stirbar was not able to rotate freely at the bottom of the resin kettle and the stirring was therefore stopped. Purging with argon was continued at a reduced flow rate (0.2 bar).

The temperature was monitored; typically, it rises from about 20° C. to about 80° C. within 60 minutes. Once the temperature starts to drop from a maximal value, the resin kettle was transferred into a circulation oven (e.g. Binder FED 720 from Binder GmbH) and kept at about 60° C. for about 18 hours.

After this time, the oven was switched off and the resin kettle was allowed to cool down for about 2 hours while remaining in the oven. After that, the gel was removed and broken manually or cut with scissors into smaller pieces. The gel was grinded with a grinder (X70G from Scharfen Slicing Machines GmbH with Unger R70 plate system: 3 pre-cutter kidney plates with straight holes at 17 mm diameter), put onto perforated stainless steel dishes (hole diameter 4.8 mm, 50 cm×50 cm, 0.55 mm caliper, 50% open area, from RS; max. height of gel before drying: about 3 cm) and transferred into a circulation oven (Binder FED 720 from Binder GmbH) at about 120° C. for about 20 hours.

The residual moisture content of the dried gel was about 3% by weight (see UPM test method for description of how to determine moisture content).

The dried gel was then ground using a centrifuge mill (Retsch ZM 200 from Retsch GmbH with vibratory feeder DR 100 (setting 50-60), interchangeable sieve with 1.5 mm opening settings, rotary speed 8000 rpm). The milled polymer was then sieved via a sieving machine (AS 400 control from Retsch with sieves DIN/ISO 3310-1 at about 250 rpm for about for 5-10 min) to the following particle size cuts with the following yields:

| | fines | Collected fraction | crude |
|---|---|---|---|
| sieved fraction | <150 μm | 150-710 μm | >710 μm |
| yield | | ~2100 g | |

The fractions “fines” and “crude” have been discarded and not used further.

### Preparation of Base Polymer BP C5 of Comparative Example C5

A 2,000 ml resin kettle (equipped with a four-necked glass cover closed with septa, suited for the introduction of a thermometer, syringe needles) was placed into an ice bath filled with about 1 liter of water, 100 g of sodium chloride and about 200 g of ice such that the mixture covers about half the height of the resin kettle. The resin kettle was charged with about 80.0 g of solution comprising aqueous polyacrylic acid (PAA) of about 35% w concentration wherein the weight average molecular weight Mw determined by Gel Permeation Chromatography reported by size exclusion chromatography was 223 kDa (test method as described herein above). About 496.6 g water was added as ice prepared from DI water and DI water of weight about 497.5 g was also added to the mixture. A magnetic stirrer, capable of mixing the whole content, was added and stirring was started.

As the PAA was fully dispersed, the full amount of 432.5 g of glacial AA (=acrylic acid) was added to the PAA solution in the resin kettle while stirring was continued.

About 13.6 g of deionized water was taken to dissolve 0.4874 g of “KPS” (=potassium peroxydisulfate, from Sigma Aldrich) e.g. in a glass vial of 40 mL volume. The vessel with the “KPS” solution was closed with a plastic snap-on cap and set aside.

About 10.0 g of deionized water was taken to dissolve 0.0529 g of “ASC” (=Ascorbic Acid, from Sigma Aldrich) e.g. in a glass vial of 40 mL volume. The vessel with the “ASC” solution was closed with a plastic snap-on cap and set aside.

About 115 g of deionized water was taken to dissolve 3.15 g of “PEG700-DA” (=polyethylene glycol diacrylate of Mn˜700 Da from Sigma Aldrich) e.g. in a 250 mL glass beaker. The beaker with the “PEG700-DA” solution was covered e.g. with parafilm and set aside.

The remaining 3.60 g of water up to a final weight of 1136.3 g was added to the resin kettle and stirring was continued as a homogeneous solution was obtained within 1-5 minutes.

A thermometer was introduced and in total 347.6 g of 50% w NaOH (sodium hydroxide) solution (for analysis, from Merck KGaA) were added subsequently in portions such that the temperature was below 30° C.

The “PEG700-DA” solution was added to the mixture of PAA, AA and NaOH solution at a temperature below 30° C. while stirring was continued.

Then, the resin kettle was closed, the ice bath underneath removed, and a pressure relief was provided e.g. by puncturing two syringe needles through the septa. The solution was then purged vigorously with argon via an 80 cm injection needle at about 0.4 bar while stirring at about 400 rpm. The argon stream was placed close to the stirrer for efficient and fast removal of dissolved oxygen.

After about min 10 min to 1 hour of Argon purging and stirring, about 0.03 g (about 1-2 droplets) of 1% w aqueous solution of hydrogen peroxide H2O2 (Sigma-Aldrich) was added via 1 mL plastic pipette to the “KPS” solution, and the latter was then added to the reaction mixture via plastic funnel inserted temporarily in one of the resin kettle cover necks while stirring and Argon purging was continued. Thereafter the “ASC” solution was added to the reaction mixture at a temperature of about 20° C. via plastic funnel inserted temporarily in one of the resin kettle cover necks while stirring and Argon purging was continued.

After the initiator solutions “KPS” and “ASC” were mixed with the reaction mixture, stirring and Argon purging was continued but the Argon needle was pulled a few cm above the liquid. Typically, within 3 min of “ASC” solution, the solution characteristically starts to become turbid or a sudden increase in viscosity was observed, typically at temperatures about room temperature. A “gel point” was observed and recorded when the stirbar was not able to rotate freely at the bottom of the resin kettle and the stirring was therefore stopped. Purging with argon was continued at a reduced flow rate (0.2 bar).

The temperature was monitored; typically, it rises from about 20° C. to about 70° C. within 60 minutes. Once the temperature starts to drop from a maximal value, the resin kettle was transferred into a circulation oven (e.g. Binder FED 720 from Binder GmbH) and kept at about 60° C. for about 18 hours.

After this time, the oven was switched off and the resin kettle was allowed to cool down for about 2 hours while remaining in the oven. After that, the gel was removed and broken manually or cut with scissors into smaller pieces. The gel was grinded with a grinder (X70G from Scharfen Slicing Machines GmbH with Unger R70 plate system: 3 pre-cutter kidney plates with straight holes at 17 mm diameter), put onto perforated stainless steel dishes (hole diameter 4.8 mm, 50 cm×50 cm, 0.55 mm caliper, 50% open area, from RS; max. height of gel before drying: about 3 cm) and transferred into a circulation oven (Binder FED 720 from Binder GmbH) at about 120° C. for about 20 hours.

The residual moisture content of the dried gel was below about 3% by weight (see UPM test method for description of how to determine moisture content).

The dried gel was then ground using a centrifuge mill (Retsch ZM 200 from Retsch GmbH with vibratory feeder DR 100 (setting 50-60), interchangeable sieve with 1.5 mm opening settings, rotary speed 8000 rpm). The milled polymer was then sieved via a sieving machine (AS 400 control from Retsch with sieves DIN/ISO 3310-1 at about 250 rpm for about for 5-10 min) to the following particle size cuts with the following yields:

| | fines | Collected fraction | crude |
|---|---|---|---|
| sieved fraction | <150 μm | 150-710 μm | >710 μm |
| yield | | ~350 g | |

The fractions “fines” and “crude” have been discarded and not used further.

### Preparation of Base Polymer BP C6 of Comparative Example C6

A 10,000 ml resin kettle (equipped with a four-necked glass cover closed with septa, suited for the introduction of a thermometer, syringe needles) was charged with about 2536.1 g of ice (ca. 60% of the total amount of ice: 3050.3 g ice prepared from deionized water). A magnetic stirrer, capable of mixing the whole content (when liquid), was added and stirring was started.

About 100.0 g of deionized water was taken to dissolve 2.599 g of “KPS” (=potassium peroxydisulfate, from Sigma Aldrich) e.g. in a glass beaker of 250 mL volume. The vessel with the “KPS” solution was closed and set aside.

About 10.0 g of deionized water was taken to dissolve 0.566 g of “ASC” (=Ascorbic Acid, from Sigma Aldrich) e.g. in a glass vial of 40 mL volume. The vessel with the “ASC” solution was closed with a plastic snap-on cap and set aside.

200.0 g of deionized water was taken to dissolve 16.76 g of “PEG700-DA” (=polyethylene glycol diacrylate of Mn˜700 Da from Sigma Aldrich) e.g. in a glass beaker. The beaker with the “PEG700-DA” solution was covered e.g. with parafilm and set aside.

The full amount of 2300.1 g of glacial AA (=acrylic acid) was added to the ice in the resin kettle while stirring was continued.

An amount of 908.7 g of Sokalan® PA 110 S (BASF) comprising aqueous polyacrylic acid solution of about 35% w concentration wherein the weight average molecular weight Mw determined by Gel Permeation Chromatography reported by size exclusion chromatography was 223 kDa (test method as described herein above), was added to the mixture in the resin kettle while stirring was continued.

A thermometer was introduced and in total 1975.4 g of 50% w NaOH (sodium hydroxide) solution (for analysis, from Merck KGaA) and the remaining amount of ice (prepared from de-ionized water) were added subsequently in portions such that the temperature was below 30° C.

The “PEG700-DA” solution was added to the mixture of AA, NaOH solution and ice at a temperature below 30° C. while stirring was continued. The beaker that contained the “PEG700-DA” solution was washed 2× with deionized water in an amount of about 10% of the “PEG700-DA” solution volume per wash. The wash water of both washing steps was added to the stirred mixture.

Deionized water (the remaining amount required to achieve the total amount of (ice+water) of 4795.0 g was added to the stirred mixture.

Then, the resin kettle was closed, and a pressure relief was provided e.g. by puncturing two syringe needles through the septa. The solution was then purged vigorously with argon via an 80 cm injection needle at about 0.4 bar while stirring at about 400 rpm. The argon stream was placed close to the stirrer for efficient and fast removal of dissolved oxygen.

After about 1 hour of Argon purging and stirring, the “ASC” solution was added to the reaction mixture at a temperature of about 20° C. via plastic funnel inserted temporarily in one of the resin kettle cover necks while stirring and Argon purging was continued. Thereafter, about 1.90 g of 1% w aqueous solution of hydrogen peroxide H2O2 (Sigma-Aldrich) was added via 1 mL plastic pipette to the “KPS” solution, and the latter was then also added to the reaction mixture via plastic funnel inserted temporarily in one of the resin kettle cover necks while stirring and Argon purging was continued.

After the initiator solutions “KPS” and “ASC” were mixed with the reaction mixture, stirring and Argon purging was continued but the Argon needle was pulled a few cm above the liquid. Typically, within 2 min of “KPS” solution addition, the solution characteristically starts to become turbid or a sudden increase in viscosity was observed, typically at temperatures about room temperature. A “gel point” was observed and recorded when the stirbar was not able to rotate freely at the bottom of the resin kettle and the stirring was therefore stopped. Purging with argon was continued at a reduced flow rate (0.2 bar).

The temperature was monitored; typically, it rises from about 20° C. to about 80° C. within 60 minutes. Once the temperature starts to drop from a maximal value, the resin kettle was transferred into a circulation oven (e.g. Binder FED 720 from Binder GmbH) and kept at about 60° C. for about 18 hours.

After this time, the oven was switched off and the resin kettle was allowed to cool down for about 2 hours while remaining in the oven. After that, the gel was removed and broken manually or cut with scissors into smaller pieces. The gel was grinded with a grinder (X70G from Scharfen Slicing Machines GmbH with Unger R70 plate system: 3 pre-cutter kidney plates with straight holes at 17 mm diameter), put onto perforated stainless steel dishes (hole diameter 4.8 mm, 50 cm×50 cm, 0.55 mm caliper, 50% open area, from RS; max. height of gel before drying: about 3 cm) and transferred into a circulation oven (Binder FED 720 from Binder GmbH) at about 120° C. for about 20 hours.

The residual moisture content of the dried gel was about 3% by weight (see UPM test method for description of how to determine moisture content).

The dried gel was then ground using a centrifuge mill (Retsch ZM 200 from Retsch GmbH with vibratory feeder DR 100 (setting 50-60), interchangeable sieve with 1.5 mm opening settings, rotary speed 8000 rpm). The milled polymer was then sieved via a sieving machine (AS 400 control from Retsch with sieves DIN/ISO 3310-1 at about 250 rpm for about for 5-10 min) to the following particle size cuts with the following yields:

| | fines | Collected fraction | crude |
|---|---|---|---|
| sieved fraction | <150 μm | 150-710 μm | >710 μm |
| yield | | ~2100 g | |

The fractions “fines” and “crude” have been discarded and not used further.

Procedure to obtain the PAA used in Example A1 to A9 (i.e. from PAA A1 to PAA A9) from degradation of pre-existing SAP material: Persulfate mediated degradation of pre-existing SAP material.

The pre-existing SAP material used in all examples was polyacrylic acid-based pre-existing SAP material (in the form of pre-existing SAP particles) having a capacity (CRC) of 27.6 g/g, a moisture content of 0.4%, and D50 average particle size was 398 um as measured according to ISO method 13322-2 (the Particle Size Distribution PSD was 63-710 μm). The Absorption Against Pressure (AAP) of the SAP was 25.5 g/g, as determined by the EDANA method WSP 442.2-02. In deviation from EDANA WSP 442.2-02, a pressure of 0.7 psi is applied (whereas the EDANA method specifies a pressure of only 0.3 psi).

The deionized water used below is MilliporeQ. Electrical conductivity was measured with lab conductometer COND 70 INSTRUMENT without CELL, #50010522, equipped with Cell VPT51-01 C-0.1 from XS Instruments or via LF 320/Set, #300243 equipped with TetraCon® 325 from WTW, conductivity is <160 μS/cm at 0° C. Similar equipment for measuring electrical conductivity can be used accordingly. The deionized water used in the examples represents the aqueous carrier. The actual amount of deionized water (=aqueous carrier) in the sample is indicated in Table 1 in column “m_w_total”.

Unless stated otherwise, the experimental procedure was performed in a climate conditioned room at standard conditions of 23° C.±2° C. temperature and 45%±10% relative humidity.

### Procedure

Preparation of potassium persulfate solution “KPS Solution”: The needed amount (see the table with experimental settings) of potassium persulfate (KPS) was weighed on a balance as dry salt of weight m1 g (Sigma-Aldrich, >=99.0% purity, inventory number 216224-500G). It was then added into the respective grams of deionized water (i.e. the aqueous carrier in a 1 L plastic bottle (made of HDPE, Nalgene™) as given in Table 2 below and designated as “m2”. Complete dissolution of the KPS salt is observed when no visible salt crystals remain in the solution.

Wherever Hydrogen peroxide was used (HPO), a resp. amount of “KPS Solution” and respective grams of 30 weight % HPO (a.k.a. Perhydrol, Sigma-Aldrich, inventory number 216763-500ML) as given in Table 2 below and designated as “mh” was added. The so obtained amount of of “Swelling Solution”, as given in Table 2 and designated as “ms1”, was placed in appropriately sized (2 to 5 L) plastic bottle (made of HDPE, Nalgene™).

Amount of dry pre-existing SAP material (as given in Table 2 below and designated as “mSAP” was measured on a balance into a glass beaker of 500 mL volume and put into an appropriately sized glass reactor or glass beaker (2-5 L) (e.g. resp. made by Normag GmbH or Pyrex). The resp. amount of “Swelling Solution” was added into the reactor with pre-existing SAP material quickly w/o shaking, so that the dry pre-existing SAP material swells with the fluid uniformly to a resp. swelling degree defined via x-load in grams of swelling fluid per gram of dry pre-existing SAP material (x-load shown as xL in Table 2 below).

Reactor was closed with the lid (standard lid with 4 openings all closed with rubber plugs). One syringe needle was put into one of the rubber plugs to ensure pressure equilibration during heating. When a glass beaker was used instead of a reactor, the beaker was covered with aluminum foil. A circulation oven (Model Binder FED720 from Binder GmbH) was preheated to the temperature given as “T1” in the Table 2 below. As temperature T1 was reached, the closed reactor or beaker was placed into the oven for the time period specified as “t1” in Table 2 below.

The reactor with the sample was taken from the oven to cool down. The sample was filtered through a metal sieve with the mesh of 500 μm (diameter 240 mm from “Retch”) placed on the top of plastic beaker of 2-5L volume depending on the size of the example. Filtration took about 2 hours to allow for the liquid to pass into the collecting vessel. The sample can be mixed with the spoon to improve filtration rate. The yield after filtration is given as “Y1” in Table 2 below. (see table with experimental data). The extracted polymer was a clear solution. The pre-existing SAP material was a cross-linked network of polyacrylic acid, hence the clear solution comprises substantially soluble polyacrylic acid. The sample was transferred into one or more 2 L plastic bottles for further use.

An aliquot part of the clear solution with mass ms_wet is measured via 5 ml plastic syringe into a pre-weighed 20 ml glass vial (without snap-on cap). The 20 ml vial with the clear solution is then put into a vacuum oven (Heraeus Vacutherm type, Thermo Scientific™) at 40° C. and pressure between 5 and 50 mbar for 3 hours to ensure substantial evaporation of the water. The dry polymeric residue is weighed and its mass ms_dry is used to calculate the solid content S via the formula:

$$S = ms\_dry * 100 / ms\_wet in \% w$$

TABLE 2
Preparation of “Swelling Solution” and conditions of degradation of
pre-existing SAP material for obtaining PAA A1 to PAA A9 by chemical degradation

| | | | mh(g) | | | | | | | | | |
|---|---|---|---|---|---|---|---|---|---|---|---|---|
| | | | (30% | | | | | | | | | Solid |
| | KPS, | | w | | mSA | xL, | | | | ms_wet, | ms_dry, | content |
| Example | m1(g) | m2(g) | HPO) | ms1(g) | P (g) | (g/g) | T1(° C.) | t1(h) | Y1 (g) | g | g | S (% w) |
| PAA | 2.00 | 1994.7 | 3.3 | 2000 | 200 | 10.0 | 100 | 6 | 1197.7 | 5.009 | 0.488 | 9.74 |
| A1 | | | | | | | | | | | | |
| PAA | 2.00 | 1998.0 | 0 | 2000 | 200 | 10.0 | 100 | 9 | 1448 | 2.003 | 0.201 | 10.0 |
| A2 | | | | | | | | | | | | |
| PAA | 3.00 | 2097.0 | 0 | 2100 | 300 | 7.0 | 100 | 9 | 1280 | 2.064 | 0.282 | 13.66 |
| A3 | | | | | | | | | | | | |
| PAA | 4.00 | 3996.0 | 0 | 4000 | 400 | 10.0 | 100 | 9 | 2646 | 2.036 | 0.219 | 10.78 |
| A456 | | | | | | | | | | | | |
| PAA | 4.00 | 3996.0 | 0 | 4000 | 400 | 10.0 | 115 | 10 | 3295.5 | 3.344 | 0.380 | 11.36 |
| A7 | | | | | | | | | | | | |
| PAA | 3.54 | 3493.5 | 2.93 | 3499.9 | 500 | 7.0 | 120 | 9 | 1638 | 3.624 | 0.520 | 14.34 |
| A8 | | | | | | | | | | | | |
| PAA | 3.52 | 3493.7 | 2.96 | 3500.2 | 500 | 7.0 | 120 | 16 | 2877 | 2.546 | 0.435 | 17.10 |
| A9 | | | | | | | | | | | | |

### Preparation of PAA A1-Containing Base Polymer BP A1 of Example A1

A 2,000 ml resin kettle (equipped with a four-necked glass cover closed with septa, suited for the introduction of a thermometer, syringe needles) was placed into an ice bath filled with about 1 liter of water, 100 g of sodium chloride and about 200 g of ice such that the mixture covers about half the height of the resin kettle. The resin kettle was charged with about 287.0 g of solution comprising aqueous polyacrylic acid PAA A1 obtained as described above of about 9.74% w concentration wherein the weight average molecular weight Mw determined by Gel Permeation Chromatography was 134 kDa (test method as described herein above). A magnetic stirrer, capable of mixing the whole content, was added to the resin kettle and stirring was started.

The full amount of 432.1 g of glacial AA (=acrylic acid) was added to the PAA solution in the resin kettle while stirring was continued.

About 10.0 g of deionized water was taken to dissolve 0.012 g of “ASC” (=Ascorbic Acid, from Sigma Aldrich) e.g. in a glass vial of 40 mL volume. The vessel with the “ASC” solution was closed with a plastic snap-on cap and set aside.

About 30 g of deionized water was taken to dissolve 3.15 g of “PEG700-DA” (=polyethylene glycol diacrylate of Mn˜700 Da from Sigma Aldrich) e.g. in a 50 mL glass beaker. The beaker with the “PEG700-DA” solution was covered e.g. with parafilm and set aside.

The remaining amount of water up to a final weight of 885.3 g was added to the resin kettle and stirring was continued as a homogeneous solution was obtained within 5 minutes.

A thermometer was introduced and in total 347.3 g of 50% w NaOH (sodium hydroxide) solution (for analysis, from Merck KGaA) were added subsequently in portions such that the temperature was below 30° C.

The “PEG700-DA” solution was added to the mixture of PAA, AA and NaOH solution at a temperature below 30° C. while stirring was continued.

Then, the resin kettle was closed, the ice bath underneath removed, and a pressure relief was provided e.g. by puncturing two syringe needles through the septa. The solution was then purged vigorously with argon via an 80 cm injection needle at about 0.4 bar while stirring at about 400 rpm. The argon stream was placed close to the stirrer for efficient and fast removal of dissolved oxygen.

After about minimum 10 min or up to 1 hour of Argon purging and stirring, the “ASC” solution was added to the reaction mixture at a temperature of about 20° C. via plastic funnel inserted temporarily in one of the resin kettle cover necks while stirring and Argon purging was continued.

After the initiator solution “ASC” was mixed with the reaction mixture, stirring and Argon purging was continued but the Argon needle was pulled a few cm above the liquid. Typically, within 1 min of “ASC” solution addition, the solution characteristically started to become turbid or a sudden increase in viscosity was observed, typically at temperatures about room temperature. A “gel point” was observed and recorded when the stirbar was not able to rotate freely at the bottom of the resin kettle and the stirring was therefore stopped. Purging with argon was continued at a reduced flow rate (0.2 bar).

The temperature was monitored; typically, it rises from about 20° C. to about 70° C. within 20 minutes. Once the temperature starts to drop from a maximal value, the resin kettle was transferred into a circulation oven (e.g. Binder FED 720 from Binder GmbH) and kept at about 60° C. for about 18 hours.

After this time, the oven was switched off and the resin kettle was allowed to cool down for about 2 hours while remaining in the oven. After that, the gel was removed and broken manually or cut with scissors into smaller pieces. The gel was grinded with a grinder (X70G from Scharfen Slicing Machines GmbH with Unger R70 plate system: 3 pre-cutter kidney plates with straight holes at 17 mm diameter), put onto perforated stainless steel dishes (hole diameter 4.8 mm, 50 cm×50 cm, 0.55 mm caliper, 50% open area, from RS; max. height of gel before drying: about 3 cm) and transferred into a circulation oven (Binder FED 720 from Binder GmbH) at about 120° C. for about 20 hours.

The residual moisture content of the dried gel was about 3% by weight (see UPM test method for description of how to determine moisture content).

The dried gel was then ground using a centrifuge mill (Retsch ZM 200 from Retsch GmbH with vibratory feeder DR 100 (setting 50-60), interchangeable sieve with 1.5 mm opening settings, rotary speed 8000 rpm). The milled polymer was then sieved via a sieving machine (AS 400 control from Retsch with sieves DIN/ISO 3310-1 at about 250 rpm for about for 5-10 min) to the following particle size cuts with the following yields:

| | fines | Collected fraction | crude |
|---|---|---|---|
| sieved fraction | <150 μm | 150-710 μm | >710 μm |
| yield | | ~350 g | |

The fractions “fines” and “crude” have been discarded and not used further.

### Preparation of PAA A2-Containing Base Polymer BP A2 of Example A2

A 2,000 ml resin kettle (equipped with a four-necked glass cover closed with septa, suited for the introduction of a thermometer, syringe needles) was placed into an ice bath filled with about 1 liter of water, 100 g of sodium chloride and about 200 g of ice such that the mixture covers about half the height of the resin kettle. The resin kettle was charged with about 274.7 g of solution comprising aqueous polyacrylic acid PAA A2 obtained as described above of about 10.0% w concentration wherein the weight average molecular weight Mw determined by Gel Permeation Chromatography was 277 Da (test method as described herein above). A magnetic stirrer, capable of mixing the whole content, was added to the resin kettle and stirring was started.

The full amount of 432.0 g of glacial AA (=acrylic acid) was added to the PAA solution in the resin kettle while stirring was continued.

About 20.0 g of deionized water was taken to dissolve 0.486 g of “KPS” (=potassium peroxydisulfate, from Sigma Aldrich) e.g. in a glass vial of 40 mL volume. The vessel with the “KPS” solution was closed with a plastic snap-on cap and set aside.

About 10.0 g of deionized water was taken to dissolve 0.012 g of “ASC” (=Ascorbic Acid, from Sigma Aldrich) e.g. in a glass vial of 40 mL volume. The vessel with the “ASC” solution was closed with a plastic snap-on cap and set aside.

About 30 g of deionized water was taken to dissolve 3.15 g of “PEG700-DA” (=polyethylene glycol diacrylate of Mn˜700 Da from Sigma Aldrich) e.g. in a 50 mL glass beaker. The beaker with the “PEG700-DA” solution was covered e.g. with parafilm and set aside.

The remaining amount of water up to a final weight of 975.2 g was added to the resin kettle and stirring was continued as a homogeneous solution was obtained within 5 minutes.

A thermometer was introduced and in total 313.6 g of 50% w NaOH (sodium hydroxide) solution (for analysis, from Merck KGaA) were added subsequently in portions such that the temperature was below 30° C.

The “PEG700-DA” solution was added to the mixture of PAA, AA and NaOH solution at a temperature below 30° C. while stirring was continued.

Then, the resin kettle was closed, the ice bath underneath removed, and a pressure relief was provided e.g. by puncturing two syringe needles through the septa. The solution was then purged vigorously with argon via an 80 cm injection needle at about 0.4 bar while stirring at about 400 rpm. The argon stream was placed close to the stirrer for efficient and fast removal of dissolved oxygen.

After about minimum 10 min or up to 1 hour of Argon purging and stirring, about 0.025 g (about 1-2 droplets) of 1% w aqueous solution of hydrogen peroxide H2O2 (Sigma-Aldrich) was added via 1 mL plastic pipette to the “KPS” solution, and the latter was then added to the reaction mixture via plastic funnel inserted temporarily in one of the resin kettle cover necks while stirring and Argon purging was continued. Thereafter the “ASC” solution was added to the reaction mixture at a temperature of about 20° C. via plastic funnel inserted temporarily in one of the resin kettle cover necks while stirring and Argon purging was continued.

After the initiator solutions “KPS” and “ASC” were mixed with the reaction mixture, stirring and Argon purging was continued but the Argon needle was pulled a few cm above the liquid. Typically, within 6 min of “ASC” solution addition, the solution characteristically starts to become turbid or a sudden increase in viscosity was observed, typically at temperatures about room temperature. A “gel point” was observed and recorded when the stirbar was not able to rotate freely at the bottom of the resin kettle and the stirring was therefore stopped. Purging with argon was continued at a reduced flow rate (0.2 bar).

The temperature was monitored; typically, it rises from about 20° C. to about 80° C. within 60 minutes. Once the temperature starts to drop from a maximal value, the resin kettle was transferred into a circulation oven (e.g. Binder FED 720 from Binder GmbH) and kept at about 60° C. for about 18 hours.

After this time, the oven was switched off and the resin kettle was allowed to cool down for about 2 hours while remaining in the oven. After that, the gel was removed and broken manually or cut with scissors into smaller pieces. The gel was grinded with a grinder (X70G from Scharfen Slicing Machines GmbH with Unger R70 plate system: 3 pre-cutter kidney plates with straight holes at 17 mm diameter), put onto perforated stainless steel dishes (hole diameter 4.8 mm, 50 cm×50 cm, 0.55 mm caliper, 50% open area, from RS; max. height of gel before drying: about 3 cm) and transferred into a circulation oven (Binder FED 720 from Binder GmbH) at about 120° C. for about 20 hours.

The residual moisture content of the dried gel was about 3% by weight (see UPM test method for description of how to determine moisture content).

The dried gel was then ground using a centrifuge mill (Retsch ZM 200 from Retsch GmbH with vibratory feeder DR 100 (setting 50-60), interchangeable sieve with 1.5 mm opening settings, rotary speed 8000 rpm). The milled polymer was then sieved via a sieving machine (AS 400 control from Retsch with sieves DIN/ISO 3310-1 at about 250 rpm for about for 5-10 min) to the following particle size cuts with the following yields:

| | fines | Collected fraction | crude |
|---|---|---|---|
| sieved fraction | <150 μm | 150-710 μm | >710 μm |
| yield | | ~350 g | |

The fractions “fines” and “crude” have been discarded and not used further.

### Preparation of PAA A3-Containing Base Polymer BP A3 of Example A3

A 2,000 ml resin kettle (equipped with a four-necked glass cover closed with septa, suited for the introduction of a thermometer, syringe needles) was placed into an ice bath filled with about 1 liter of water, 100 g of sodium chloride and about 200 g of ice such that the mixture covers about half the height of the resin kettle. The resin kettle was charged with about 811.0 g of solution comprising aqueous polyacrylic acid PAA A3 obtained as described above of about 6.67% w concentration wherein the weight average molecular weight Mw determined by Gel Permeation Chromatography was 517,500 Da (test method as described herein above). The 6.67% w aqueous solution of PAA A3 was prepared as a stock solution by diluting and stirring overnight of PAA A3 solution of 13.66% w concentration with the appropriate amount of DI water. A magnetic stirrer, capable of mixing the whole content (when liquid), was added to the resin kettle and stirring was started.

The full amount of 405.9 g of glacial AA (=acrylic acid) was added to the PAA solution in the resin kettle while stirring was continued.

About 20.0 g of deionized water was taken to dissolve 0.455 g of “KPS” (=potassium peroxydisulfate, from Sigma Aldrich) e.g. in a glass vial of 40 mL volume. The vessel with the “KPS” solution was closed with a plastic snap-on cap and set aside.

About 10.0 g of deionized water was taken to dissolve 0.011 g of “ASC” (=Ascorbic Acid, from Sigma Aldrich) e.g. in a glass vial of 40 mL volume. The vessel with the “ASC” solution was closed with a plastic snap-on cap and set aside.

About 30 g of deionized water was taken to dissolve 2.95 g of “PEG700-DA” (=polyethylene glycol diacrylate of Mn˜700 Da from Sigma Aldrich) e.g. in a 50 mL glass beaker. The beaker with the “PEG700-DA” solution was covered e.g. with parafilm and set aside.

The remaining amount of water up to a final weight of 497.7 g was added to the resin kettle and stirring was continued as a homogeneous solution was obtained within 1-5 minutes.

A thermometer was introduced and in total 281.8 g of 50% w NaOH (sodium hydroxide) solution (for analysis, from Merck KGAA) were added subsequently in portions such that the temperature was below 30° C.

The “PEG700-DA” solution was added to the mixture of PAA, AA and NaOH solution at a temperature below 30° C. while stirring was continued.

Then, the resin kettle was closed, the ice bath underneath removed, and a pressure relief was provided e.g. by puncturing two syringe needles through the septa. The solution was then purged vigorously with argon via an 80 cm injection needle at about 0.4 bar while stirring at about 400 rpm. The argon stream was placed close to the stirrer for efficient and fast removal of dissolved oxygen.

After about minimum 10 min or up to 1 hour of Argon purging and stirring, about 0.025 g (about 1-2 droplets) of 1% w aqueous solution of hydrogen peroxide H2O2 (Sigma-Aldrich) was added via 1 mL plastic pipette to the “KPS” solution, and the latter was then added to the reaction mixture via plastic funnel inserted temporarily in one of the resin kettle cover necks while stirring and Argon purging was continued. Thereafter the “ASC” solution was added to the reaction mixture at a temperature of about 20° C. via plastic funnel inserted temporarily in one of the resin kettle cover necks while stirring and Argon purging was continued.

After the initiator solutions “KPS” and “ASC” were mixed with the reaction mixture, stirring and Argon purging was continued but the Argon needle was pulled a few cm above the liquid. Typically, within 4 min of “ASC” solution addition, the solution characteristically starts to become turbid or a sudden increase in viscosity was observed, typically at temperatures about room temperature. A “gel point” was observed and recorded when the stirbar was not able to rotate freely at the bottom of the resin kettle and the stirring was therefore stopped. Purging with argon was continued at a reduced flow rate (0.2 bar).

The temperature was monitored; typically, it rises from about 20° C. to about 70° C. within 60 minutes. Once the temperature starts to drop from a maximal value, the resin kettle was transferred into a circulation oven (e.g. Binder FED 720 from Binder GmbH) and kept at about 60° C. for about 18 hours.

After this time, the oven was switched off and the resin kettle was allowed to cool down for about 2 hours while remaining in the oven. After that, the gel was removed and broken manually or cut with scissors into smaller pieces. The gel was grinded with a grinder (X70G from Scharfen Slicing Machines GmbH with Unger R70 plate system: 3 pre-cutter kidney plates with straight holes at 17 mm diameter), put onto perforated stainless steel dishes (hole diameter 4.8 mm, 50 cm×50 cm, 0.55 mm caliper, 50% open area, from RS; max. height of gel before drying: about 3 cm) and transferred into a circulation oven (Binder FED 720 from Binder GmbH) at about 120° C. for about 20 hours.

The residual moisture content of the dried gel was about 3% by weight (see UPM test method for description of how to determine moisture content).

The dried gel was then ground using a centrifuge mill (Retsch ZM 200 from Retsch GmbH with vibratory feeder DR 100 (setting 50-60), interchangeable sieve with 1.5 mm opening settings, rotary speed 8000 rpm). The milled polymer was then sieved via a sieving machine (AS 400 control from Retsch with sieves DIN/ISO 3310-1 at about 250 rpm for about for 5-10 min) to the following particle size cuts with the following yields:

| | fines | Collected fraction | crude |
|---|---|---|---|
| sieved fraction | <150 μm | 150-710 μm | >710 μm |
| yield | | ~350 g | |

The fractions “fines” and “crude” have been discarded and not used further.

### Preparation of PAA A456-Containing Base Polymer BP A4 of Example A4

A 2,000 ml resin kettle (equipped with a four-necked glass cover closed with septa, suited for the introduction of a thermometer, syringe needles) was placed into an ice bath filled with about 1 liter of water, 100 g of sodium chloride and about 200 g of ice such that the mixture covers about half the height of the resin kettle. The resin kettle was charged with about 466.3 g of solution comprising aqueous polyacrylic acid PAA A456 obtained as described above of about 10.78% w concentration wherein the weight average molecular weight Mw determined by Gel Permeation Chromatography was 285 Da (test method as described herein above). A magnetic stirrer, capable of mixing the whole content, was added to the resin kettle and stirring was started.

The full amount of 380.2 g of glacial AA (=acrylic acid) was added to the PAA solution in the resin kettle while stirring was continued.

About 20.0 g of deionized water was taken to dissolve 0.431 g of “KPS” (=potassium peroxydisulfate, from Sigma Aldrich) e.g. in a glass vial of 40 mL volume. The vessel with the “KPS” solution was closed with a plastic snap-on cap and set aside.

About 10.0 g of deionized water was taken to dissolve 0.047 g of “ASC” (=Ascorbic Acid, from Sigma Aldrich) e.g. in a glass vial of 40 mL volume. The vessel with the “ASC” solution was closed with a plastic snap-on cap and set aside.

About 30 g of deionized water was taken to dissolve 2.77 g of “PEG700-DA” (=polyethylene glycol diacrylate of Mn˜700 Da from Sigma Aldrich) e.g. in a 50 mL glass beaker. The beaker with the “PEG700-DA” solution was covered e.g. with parafilm and set aside.

The remaining amount of water up to a final weight of 899.6 g was added to the resin kettle and stirring was continued as a homogeneous solution was obtained within 5 minutes.

A thermometer was introduced and in total 250.8 g of 50% w NaOH (sodium hydroxide) solution (for analysis, from Merck KGaA) were added subsequently in portions such that the temperature was below 30° C.

The “PEG700-DA” solution was added to the mixture of PAA, AA and NaOH solution at a temperature below 30° C. while stirring was continued.

Then, the resin kettle was closed, the ice bath underneath removed, and a pressure relief was provided e.g. by puncturing two syringe needles through the septa. The solution was then purged vigorously with argon via an 80 cm injection needle at about 0.4 bar while stirring at about 400 rpm. The argon stream was placed close to the stirrer for efficient and fast removal of dissolved oxygen.

After about minimum 10 min of Argon purging and stirring, about 0.025 g (about 1-2 droplets) of 1% w aqueous solution of hydrogen peroxide H2O2 (Sigma-Aldrich) was added via 1 mL plastic pipette to the “KPS” solution, and the latter was then added to the reaction mixture via plastic funnel inserted temporarily in one of the resin kettle cover necks while stirring and Argon purging was continued. Thereafter the “ASC” solution was added to the reaction mixture at a temperature of about 20° C. via plastic funnel inserted temporarily in one of the resin kettle cover necks while stirring and Argon purging was continued.

After the initiator solutions “KPS” and “ASC” were mixed with the reaction mixture, stirring and Argon purging was continued but the Argon needle was pulled a few cm above the liquid. Typically, within 13 min of “ASC” solution addition, the solution characteristically starts to become turbid or a sudden increase in viscosity was observed, typically at temperatures about room temperature. A “gel point” was observed and recorded when the stirbar was not able to rotate freely at the bottom of the resin kettle and the stirring was therefore stopped. Purging with argon was continued at a reduced flow rate (0.2 bar).

The temperature was monitored; typically, it rises from about 20° C. to about 60° C. within 90 minutes. Once the temperature starts to drop from a maximal value, the resin kettle was transferred into a circulation oven (e.g. Binder FED 720 from Binder GmbH) and kept at about 60° C. for about 18 hours.

After this time, the oven was switched off and the resin kettle was allowed to cool down for about 2 hours while remaining in the oven. After that, the gel was removed and broken manually or cut with scissors into smaller pieces. The gel was grinded with a grinder (X70G from Scharfen Slicing Machines GmbH with Unger R70 plate system: 3 pre-cutter kidney plates with straight holes at 17 mm diameter), put onto perforated stainless steel dishes (hole diameter 4.8 mm, 50 cm×50 cm, 0.55 mm caliper, 50% open area, from RS; max. height of gel before drying: about 3 cm) and transferred into a circulation oven (Binder FED 720 from Binder GmbH) at about 120° C. for about 20 hours.

The residual moisture content of the dried gel was about 3% by weight (see UPM test method for description of how to determine moisture content).

The dried gel was then ground using a centrifuge mill (Retsch ZM 200 from Retsch GmbH with vibratory feeder DR 100 (setting 50-60), interchangeable sieve with 1.5 mm opening settings, rotary speed 8000 rpm). The milled polymer was then sieved via a sieving machine (AS 400 control from Retsch with sieves DIN/ISO 3310-1 at about 250 rpm for about for 5-10 min) to the following particle size cuts with the following yields:

| | fines | Collected fraction | crude |
|---|---|---|---|
| sieved fraction | <150 μm | 150-710 μm | >710 μm |
| yield | | ~350 g | |

The fractions “fines” and “crude” have been discarded and not used further.

### Preparation of PAA A456-Containing Base Polymer BP A5 of Example A5

A 2,000 ml resin kettle (equipped with a four-necked glass cover closed with septa, suited for the introduction of a thermometer, syringe needles) was placed into an ice bath filled with about 1 liter of water, 100 g of sodium chloride and about 200 g of ice such that the mixture covers about half the height of the resin kettle. The resin kettle was charged with about 1413.9 g of solution comprising aqueous polyacrylic acid PAA A456 obtained as described above of about 10.78% w concentration wherein the weight average molecular weight Mw determined by Gel Permeation Chromatography was 285 Da (test method as described herein above). A magnetic stirrer, capable of mixing the whole content, was added to the resin kettle and stirring was started.

The full amount of 380.0 g of glacial AA (=acrylic acid) was added to the PAA solution in the resin kettle while stirring was continued.

About 20.0 g of deionized water was taken to dissolve 0.348 g of “KPS” (=potassium peroxydisulfate, from Sigma Aldrich) e.g. in a glass vial of 40 mL volume. The vessel with the “KPS” solution was closed with a plastic snap-on cap and set aside.

About 10.0 g of deionized water was taken to dissolve 0.085 g of “ASC” (=Ascorbic Acid, from Sigma Aldrich) e.g. in a glass vial of 40 mL volume. The vessel with the “ASC” solution was closed with a plastic snap-on cap and set aside.

About 30 g of deionized water was taken to dissolve 1.20 g of “PEG700-DA” (=polyethylene glycol diacrylate of Mn˜700 Da from Sigma Aldrich) e.g. in a 50 mL glass beaker. The beaker with the “PEG700-DA” solution was covered e.g. with parafilm and set aside.

The remaining amount of water up to a final weight of 137.2 g was added to the resin kettle and stirring was continued as a homogeneous solution was obtained within 5 minutes.

A thermometer was introduced and in total 167.6 g of 50% w NaOH (sodium hydroxide) solution (for analysis, from Merck KGaA) were added subsequently in portions such that the temperature was below 30° C.

The “PEG700-DA” solution was added to the mixture of PAA, AA and NaOH solution at a temperature below 30° C. while stirring was continued.

Then, the resin kettle was closed, the ice bath underneath removed, and a pressure relief was provided e.g. by puncturing two syringe needles through the septa. The solution was then purged vigorously with argon via an 80 cm injection needle at about 0.4 bar while stirring at about 400 rpm. The argon stream was placed close to the stirrer for efficient and fast removal of dissolved oxygen.

After about minimum 10 min of Argon purging and stirring, about 0.02 g (about 1-2 droplets) of 1% w aqueous solution of hydrogen peroxide H2O2 (Sigma-Aldrich) was added via 1 mL plastic pipette to the “KPS” solution, and the latter was then added to the reaction mixture via plastic funnel inserted temporarily in one of the resin kettle cover necks while stirring and Argon purging was continued. Thereafter the “ASC” solution was added to the reaction mixture at a temperature of about 20° C. via plastic funnel inserted temporarily in one of the resin kettle cover necks while stirring and Argon purging was continued.

After the initiator solutions “KPS” and “ASC” were mixed with the reaction mixture, stirring and Argon purging was continued but the Argon needle was pulled a few cm above the liquid. Typically, within 4 min of “ASC” solution addition, the solution characteristically starts to become turbid or a sudden increase in viscosity was observed, typically at temperatures about room temperature. A “gel point” was observed and recorded when the stirbar was not able to rotate freely at the bottom of the resin kettle and the stirring was therefore stopped. Purging with argon was continued at a reduced flow rate (0.2 bar).

The temperature was monitored; typically, it rises from about 20° C. to about 35° C. within 90 minutes. Once the temperature started to drop from a maximal value, the resin kettle was transferred into a circulation oven (e.g. Binder FED 720 from Binder GmbH) and kept at about 60° C. for about 18 hours.

After this time, the oven was switched off and the resin kettle was allowed to cool down for about 2 hours while remaining in the oven. After that, the gel was removed and broken manually or cut with scissors into smaller pieces. The gel was grinded with a grinder (X70G from Scharfen Slicing Machines GmbH with Unger R70 plate system: 3 pre-cutter kidney plates with straight holes at 17 mm diameter), put onto perforated stainless steel dishes (hole diameter 4.8 mm, 50 cm×50 cm, 0.55 mm caliper, 50% open area, from RS; max. height of gel before drying: about 3 cm) and transferred into a circulation oven (Binder FED 720 from Binder GmbH) at about 120° C. for about 20 hours.

The residual moisture content of the dried gel was about 3% by weight (see UPM test method for description of how to determine moisture content).

The dried gel was then ground using a centrifuge mill (Retsch ZM 200 from Retsch GmbH with vibratory feeder DR 100 (setting 50-60), interchangeable sieve with 1.5 mm opening settings, rotary speed 8000 rpm). The milled polymer was then sieved via a sieving machine (AS 400 control from Retsch with sieves DIN/ISO 3310-1 at about 250 rpm for about for 5-10 min) to the following particle size cuts with the following yields:

| | fines | Collected fraction | crude |
|---|---|---|---|
| sieved fraction | <150 μm | 150-710 μm | >710 μm |
| yield | | ~350 g | |

The fractions “fines” and “crude” have been discarded and not used further.

### Preparation of PAA A456-Containing Base Polymer BP A6 of Example A6

A 2,000 ml resin kettle (equipped with a four-necked glass cover closed with septa, suited for the introduction of a thermometer, syringe needles) was placed into an ice bath filled with about 1 liter of water, 100 g of sodium chloride and about 200 g of ice such that the mixture covers about half the height of the resin kettle. The resin kettle was charged with about 740.5 g of solution comprising aqueous polyacrylic acid PAA A456 obtained as described above of about 10.78% w concentration wherein the weight average molecular weight Mw determined by Gel Permeation Chromatography was 285 Da (test method as described herein above). A magnetic stirrer, capable of mixing the whole content, was added to the resin kettle and stirring was started.

The full amount of 380.1 g of glacial AA (=acrylic acid) was added to the PAA solution in the resin kettle while stirring was continued.

About 20.0 g of deionized water was taken to dissolve 0.427 g of “KPS” (=potassium peroxydisulfate, from Sigma Aldrich) e.g. in a glass vial of 40 mL volume. The vessel with the “KPS” solution was closed with a plastic snap-on cap and set aside.

About 10.0 g of deionized water was taken to dissolve 0.045 g of “ASC” (=Ascorbic Acid, from Sigma Aldrich) e.g. in a glass vial of 40 mL volume. The vessel with the “ASC” solution was closed with a plastic snap-on cap and set aside.

About 30 g of deionized water was taken to dissolve 2.78 g of “PEG700-DA” (=polyethylene glycol diacrylate of Mn˜700 Da from Sigma Aldrich) e.g. in a 50 mL glass beaker. The beaker with the “PEG700-DA” solution was covered e.g. with parafilm and set aside.

The remaining amount of water up to a final weight of 625.4 g was added to the resin kettle and stirring was continued as a homogeneous solution was obtained within 5 minutes.

A thermometer was introduced and in total 250.8 g of 50% w NaOH (sodium hydroxide) solution (for analysis, from Merck KGaA) were added subsequently in portions such that the temperature was below 30° C.

The “PEG700-DA” solution was added to the mixture of PAA, AA and NaOH solution at a temperature below 30° C. while stirring was continued.

Then, the resin kettle was closed, the ice bath underneath removed, and a pressure relief was provided e.g. by puncturing two syringe needles through the septa. The solution was then purged vigorously with argon via an 80 cm injection needle at about 0.4 bar while stirring at about 400 rpm. The argon stream was placed close to the stirrer for efficient and fast removal of dissolved oxygen.

After about minimum 10 min of Argon purging and stirring, about 0.025 g (about 1-2 droplets) of 1% w aqueous solution of hydrogen peroxide H2O2 (Sigma-Aldrich) was added via 1 mL plastic pipette to the “KPS” solution, and the latter was then added to the reaction mixture via plastic funnel inserted temporarily in one of the resin kettle cover necks while stirring and Argon purging was continued. Thereafter the “ASC” solution was added to the reaction mixture at a temperature of about 20° C. via plastic funnel inserted temporarily in one of the resin kettle cover necks while stirring and Argon purging was continued.

After the initiator solutions “KPS” and “ASC” were mixed with the reaction mixture, stirring and Argon purging was continued but the Argon needle was pulled a few cm above the liquid. Typically, within 9 min of “ASC” solution addition, the solution characteristically starts to become turbid or a sudden increase in viscosity was observed, typically at temperatures about room temperature. A “gel point” was observed and recorded when the stirbar was not able to rotate freely at the bottom of the resin kettle and the stirring was therefore stopped. Purging with argon was continued at a reduced flow rate (0.2 bar).

The temperature was monitored; typically, it rises from about 20° C. to about 65° C. within 90 minutes. Once the temperature starts to drop from a maximal value, the resin kettle was transferred into a circulation oven (e.g. Binder FED 720 from Binder GmbH) and kept at about 60° C. for about 18 hours.

After this time, the oven was switched off and the resin kettle was allowed to cool down for about 2 hours while remaining in the oven. After that, the gel was removed and broken manually or cut with scissors into smaller pieces. The gel was grinded with a grinder (X70G from Scharfen Slicing Machines GmbH with Unger R70 plate system: 3 pre-cutter kidney plates with straight holes at 17 mm diameter), put onto perforated stainless steel dishes (hole diameter 4.8 mm, 50 cm×50 cm, 0.55 mm caliper, 50% open area, from RS; max. height of gel before drying: about 3 cm) and transferred into a circulation oven (Binder FED 720 from Binder GmbH) at about 120° C. for about 20 hours.

The residual moisture content of the dried gel was about 3% by weight (see UPM test method for description of how to determine moisture content).

The dried gel was then ground using a centrifuge mill (Retsch ZM 200 from Retsch GmbH with vibratory feeder DR 100 (setting 50-60), interchangeable sieve with 1.5 mm opening settings, rotary speed 8000 rpm). The milled polymer was then sieved via a sieving machine (AS 400 control from Retsch with sieves DIN/ISO 3310-1 at about 250 rpm for about for 5-10 min) to the following particle size cuts with the following yields:

| | fines | Collected fraction | crude |
|---|---|---|---|
| sieved fraction | <150 μm | 150-710 μm | >710 μm |
| yield | | ~350 g | |

The fractions “fines” and “crude” have been discarded and not used further.

### Preparation of PAA A7-Containing Base Polymer BP A7 of Example A7

A 2,000 ml resin kettle (equipped with a four-necked glass cover closed with septa, suited for the introduction of a thermometer, syringe needles) was placed into an ice bath filled with about 1 liter of water, 100 g of sodium chloride and about 200 g of ice such that the mixture covers about half the height of the resin kettle. The resin kettle was charged with about 1192.7 g of solution comprising aqueous polyacrylic acid PAA A7 obtained as described above of about 11.36% w concentration wherein the weight average molecular weight Mw determined by Gel Permeation Chromatography was 285 Da (test method as described herein above). A magnetic stirrer, capable of mixing the whole content, was added to the resin kettle and stirring was started.

The full amount of 460.1 g of glacial AA (=acrylic acid) was added to the PAA solution in the resin kettle while stirring was continued.

About 20.0 g of deionized water was taken to dissolve 0.517 g of “KPS” (=potassium peroxydisulfate, from Sigma Aldrich) e.g. in a glass vial of 40 mL volume. The vessel with the “KPS” solution was closed with a plastic snap-on cap and set aside.

About 5.0 g of deionized water was taken to dissolve 0.011 g of “ASC” (=Ascorbic Acid, from Sigma Aldrich) e.g. in a glass vial of 40 mL volume. The vessel with the “ASC” solution was closed with a plastic snap-on cap and set aside.

About 20 g of deionized water was taken to dissolve 1.78 g of “PEG700-DA” (=polyethylene glycol diacrylate of Mn˜700 Da from Sigma Aldrich) e.g. in a 50 mL glass beaker. The beaker with the “PEG700-DA” solution was covered e.g. with parafilm and set aside.

The remaining amount of water up to a final weight of 55.4 g was added to the resin kettle and stirring was continued as a homogeneous solution was obtained within 5 minutes.

A thermometer was introduced and in total 289.6 g of 50% w NaOH (sodium hydroxide) solution (for analysis, from Merck KGaA) were added subsequently in portions such that the temperature was below 30° C.

The “PEG700-DA” solution was added to the mixture of PAA, AA and NaOH solution at a temperature below 30° C. while stirring was continued.

Then, the resin kettle was closed, the ice bath underneath removed, and a pressure relief was provided e.g. by puncturing two syringe needles through the septa. The solution was then purged vigorously with argon via an 80 cm injection needle at about 0.4 bar while stirring at about 400 rpm. The argon stream was placed close to the stirrer for efficient and fast removal of dissolved oxygen.

After about minimum 10 min of Argon purging and stirring, about 0.020 g (about 1-2 droplets) of 1% w aqueous solution of hydrogen peroxide H2O2 (Sigma-Aldrich) was added via 1 mL plastic pipette to the “KPS” solution, and the latter was then added to the reaction mixture via plastic funnel inserted temporarily in one of the resin kettle cover necks while stirring and Argon purging was continued. Thereafter the “ASC” solution was added to the reaction mixture at a temperature of about 20° C. via plastic funnel inserted temporarily in one of the resin kettle cover necks while stirring and Argon purging was continued.

After the initiator solutions “KPS” and “ASC” were mixed with the reaction mixture, stirring and Argon purging was continued but the Argon needle was pulled a few cm above the liquid. Typically, within 3 min of “ASC” solution addition, the solution characteristically starts to become turbid or a sudden increase in viscosity was observed, typically at temperatures about room temperature. A “gel point” was observed and recorded when the stirbar was not able to rotate freely at the bottom of the resin kettle and the stirring was therefore stopped. Purging with argon was continued at a reduced flow rate (0.2 bar).

The temperature was monitored; typically, it rises from about 20° C. to about 70° C. within 60 minutes. Once the temperature starts to drop from a maximal value, the resin kettle was transferred into a circulation oven (e.g. Binder FED 720 from Binder GmbH) and kept at about 60° C. for about 18 hours.

After this time, the oven was switched off and the resin kettle was allowed to cool down for about 2 hours while remaining in the oven. After that, the gel was removed and broken manually or cut with scissors into smaller pieces. The gel was grinded with a grinder (X70G from Scharfen Slicing Machines GmbH with Unger R70 plate system: 3 pre-cutter kidney plates with straight holes at 17 mm diameter), put onto perforated stainless steel dishes (hole diameter 4.8 mm, 50 cm×50 cm, 0.55 mm caliper, 50% open area, from RS; max. height of gel before drying: about 3 cm) and transferred into a circulation oven (Binder FED 720 from Binder GmbH) at about 120° C. for about 20 hours.

The residual moisture content of the dried gel was about 3% by weight (see UPM test method for description of how to determine moisture content).

The dried gel was then ground using a centrifuge mill (Retsch ZM 200 from Retsch GmbH with vibratory feeder DR 100 (setting 50-60), interchangeable sieve with 1.5 mm opening settings, rotary speed 8000 rpm). The milled polymer was then sieved via a sieving machine (AS 400 control from Retsch with sieves DIN/ISO 3310-1 at about 250 rpm for about for 5-10 min) to the following particle size cuts with the following yields:

| | fines | Collected fraction | crude |
|---|---|---|---|
| sieved fraction | <150 μm | 150-710 μm | >710 μm |
| yield | | ~350 g | |

The fractions “fines” and “crude” have been discarded and not used further.

### Preparation of PAA A8-Containing Base Polymer BP A8 of Example A8

A 2,000 ml resin kettle (equipped with a four-necked glass cover closed with septa, suited for the introduction of a thermometer, syringe needles) was placed into an ice bath filled with about 1 liter of water, 100 g of sodium chloride and about 200 g of ice such that the mixture covers about half the height of the resin kettle. The resin kettle was charged with about 1085.4 g of solution comprising aqueous polyacrylic acid PAA A8 obtained as described above of about 14.34% w concentration wherein the weight average molecular weight Mw determined by Gel Permeation Chromatography was 239 Da (test method as described herein above). A magnetic stirrer, capable of mixing the whole content, was added to the resin kettle and stirring was started.

The full amount of 530.0 g of glacial AA (=acrylic acid) was added to the PAA solution in the resin kettle while stirring was continued.

About 20.0 g of deionized water was taken to dissolve 0.596 g of “KPS” (=potassium peroxydisulfate, from Sigma Aldrich) e.g. in a glass vial of 40 mL volume. The vessel with the “KPS” solution was closed with a plastic snap-on cap and set aside.

About 5.0 g of deionized water was taken to dissolve 0.013 g of “ASC” (=Ascorbic Acid, from Sigma Aldrich) e.g. in a glass vial of 40 mL volume. The vessel with the “ASC” solution was closed with a plastic snap-on cap and set aside.

No additional crosslinker “PEG700-DA” (=polyethylene glycol diacrylate of Mn˜700 Da from Sigma Aldrich) was added.

The remaining amount of water up to a final weight of 49.92 g was added to the resin kettle and stirring was continued as a homogeneous solution was obtained within 5 minutes.

A thermometer was introduced and in total 334.1 g of 50% w NaOH (sodium hydroxide) solution (for analysis, from Merck KGaA) were added subsequently in portions such that the temperature was below 30° C.

Then, the resin kettle was closed, the ice bath underneath removed, and a pressure relief was provided e.g. by puncturing two syringe needles through the septa. The solution was then purged vigorously with argon via an 80 cm injection needle at about 0.4 bar while stirring at about 400 rpm. The argon stream was placed close to the stirrer for efficient and fast removal of dissolved oxygen.

After about minimum 10 min of Argon purging and stirring, about 0.030 g (about 2 droplets) of 1% w aqueous solution of hydrogen peroxide H2O2 (Sigma-Aldrich) was added via 1 mL plastic pipette to the “KPS” solution, and the latter was then added to the reaction mixture via plastic funnel inserted temporarily in one of the resin kettle cover necks while stirring and Argon purging was continued. Thereafter the “ASC” solution was added to the reaction mixture at a temperature of about 20° C. via plastic funnel inserted temporarily in one of the resin kettle cover necks while stirring and Argon purging was continued.

After the initiator solutions “KPS” and “ASC” were mixed with the reaction mixture, stirring and Argon purging was continued but the Argon needle was pulled a few cm above the liquid. Typically, within 4 min of “ASC” solution addition, the solution characteristically starts to become turbid or a sudden increase in viscosity was observed, typically at temperatures about room temperature. A “gel point” was observed and recorded when the stirbar was not able to rotate freely at the bottom of the resin kettle and the stirring was therefore stopped. Purging with argon was continued at a reduced flow rate (0.2 bar).

The temperature was monitored; typically, it rises from about 20° C. to about 100° C. within 20 minutes. Once the temperature starts to drop from a maximal value, the resin kettle was transferred into a circulation oven (e.g. Binder FED 720 from Binder GmbH) and kept at about 60° C. for about 18 hours.

After this time, the oven was switched off and the resin kettle was allowed to cool down for about 2 hours while remaining in the oven. After that, the gel was removed and broken manually or cut with scissors into smaller pieces. The gel was grinded with a grinder (X70G from Scharfen Slicing Machines GmbH with Unger R70 plate system: 3 pre-cutter kidney plates with straight holes at 17 mm diameter), put onto perforated stainless steel dishes (hole diameter 4.8 mm, 50 cm×50 cm, 0.55 mm caliper, 50% open area, from RS; max. height of gel before drying: about 3 cm) and transferred into a circulation oven (Binder FED 720 from Binder GmbH) at about 120° C. for about 20 hours.

The residual moisture content of the dried gel was about 3% by weight (see UPM test method for description of how to determine moisture content).

The dried gel was then ground using a centrifuge mill (Retsch ZM 200 from Retsch GmbH with vibratory feeder DR 100 (setting 50-60), interchangeable sieve with 1.5 mm opening settings, rotary speed 8000 rpm). The milled polymer was then sieved via a sieving machine (AS 400 control from Retsch with sieves DIN/ISO 3310-1 at about 250 rpm for about for 5-10 min) to the following particle size cuts with the following yields:

| | fines | Collected fraction | crude |
|---|---|---|---|
| sieved fraction | <150 μm | 150-710 μm | >710 μm |
| yield | | ~350 g | |

The fractions “fines” and “crude” have been discarded and not used further.

### Preparation of PAA A9-Containing Base Polymer BP A9 of Example A9

A 2,000 ml resin kettle (equipped with a four-necked glass cover closed with septa, suited for the introduction of a thermometer, syringe needles) was charged with about 896.7 g of solution comprising aqueous polyacrylic acid PAA A9 obtained as described above of about 17.10% w concentration wherein the weight average molecular weight Mw determined by Gel Permeation Chromatography was 229 Da (test method as described herein above). A magnetic stirrer, capable of mixing the whole content, was added to the resin kettle and stirring was started.

The full amount of 76.6 g of glacial AA (=acrylic acid) was added to the PAA solution in the resin kettle while stirring was continued.

About 10.0 g of deionized water was taken to dissolve 0.086 g of “KPS” (=potassium peroxydisulfate, from Sigma Aldrich) e.g. in a glass vial of 40 mL volume. The vessel with the “KPS” solution was closed with a plastic snap-on cap and set aside.

About 5.0 g of deionized water was taken to dissolve 0.020 g of “ASC” (=Ascorbic Acid, from Sigma Aldrich) e.g. in a glass vial of 40 mL volume. The vessel with the “ASC” solution was closed with a plastic snap-on cap and set aside.

No additional crosslinker “PEG700-DA” (=polyethylene glycol diacrylate of Mn˜700 Da from Sigma Aldrich) was added.

The remaining amount of water up to a final weight of 26.83 g was added to the resin kettle and stirring was continued as a homogeneous solution was obtained within 5 minutes.

A thermometer was introduced. No further NaOH (sodium hydroxide) solution was added subsequently.

Then, the resin kettle was closed, and a pressure relief was provided e.g. by puncturing two syringe needles through the septa. The solution was then purged vigorously with argon via an 80 cm injection needle at about 0.4 bar while stirring at about 400 rpm. The argon stream was placed close to the stirrer for efficient and fast removal of dissolved oxygen.

After about minimum 10 min of Argon purging and stirring, the “ASC” solution was added to the “KPS” solution, and in turn the resulting mixture was then added to the reaction mixture via plastic funnel inserted temporarily in one of the resin kettle cover necks while stirring and Argon purging was continued. The reaction mixture was at a temperature of about 20° C.

After the initiator solution “KPS” (and therein the “ASC” solution), was mixed with the reaction mixture, stirring and Argon purging was continued but the Argon needle was pulled a few cm above the liquid. Typically, within 2 min of “ASC” solution addition, the solution characteristically became turbid or a sudden increase in viscosity was observed, typically at temperatures about room temperature. A “gel point” was observed and recorded when the stirbar was not able to rotate freely at the bottom of the resin kettle and the stirring was therefore stopped. Purging with argon was continued at a reduced flow rate (0.2 bar).

The temperature was monitored; it rose slightly from about 20° C. to about 31° C. within 20 minutes. Once the temperature started to drop from a maximal value, the resin kettle was transferred into a circulation oven (e.g. Binder FED 720 from Binder GmbH) and kept at about 60° C. for about 18 hours.

After this time, the oven was switched off and the resin kettle was allowed to cool down for about 2 hours while remaining in the oven. After that, the gel was removed and broken manually or cut with scissors into smaller pieces. The gel was grinded with a grinder (X70G from Scharfen Slicing Machines GmbH with Unger R70 plate system: 3 pre-cutter kidney plates with straight holes at 17 mm diameter), put onto perforated stainless steel dishes (hole diameter 4.8 mm, 50 cm×50 cm, 0.55 mm caliper, 50% open area, from RS; max. height of gel before drying: about 3 cm) and transferred into a circulation oven (Binder FED 720 from Binder GmbH) at about 120° C. for about 20 hours.

The residual moisture content of the dried gel was about 3% by weight (see UPM test method for description of how to determine moisture content).

The dried gel was then ground using a centrifuge mill (Retsch ZM 200 from Retsch GmbH with vibratory feeder DR 100 (setting 50-60), interchangeable sieve with 1.5 mm opening settings, rotary speed 8000 rpm). The milled polymer was then sieved via a sieving machine (AS 400 control from Retsch with sieves DIN/ISO 3310-1 at about 250 rpm for about for 5-10 min) to the following particle size cuts with the following yields:

| | fines | Collected fraction | crude |
|---|---|---|---|
| sieved fraction | <150 μm | 150-710 μm | >710 μm |
| yield | | ~350 g | |

The fractions “fines” and “crude” have been discarded and not used further.

Procedure to obtain the PAA used in Example A10 (=PAA A10) from degradation of pre-existing SAP material: Ultraviolet light mediated degradation of pre-existing SAP material

The pre-existing SAP material (in the form of pre-existing SAP particles) used for degradation is commercially available in Pampers Baby Dry as marketed in Germany in 2020.

The pre-existing SAP material was mixed with RO (reverse osmosis) water in a Quadro mixer to produce a feed stream (in the form of a gel) with 2.5% wt SAP and 97.5% RO water. Starting viscosity of the gel was around 840 Pa·s. About 140 mL of the feed stream was loaded in a syringe and fed into a Fusion UV Curing system (FUSION UV SYSTEMS, Inc., Maryland, USA; Hg lamp (H-Bulb) with 300 W/in. and 2.74 W/cm2 power measured by the UV PowerMAP® #20082105 A/B/C/V (EIT, Inc.; Sterling, VA)) in a 6 mm external diameter (OD) (3.68 mm internal diameter (ID)) quartz tube and at a rate of 6 mL/min using a syringe pump (New Era Pump Systems, Inc., Farmingdale, NY; model NE-1000 single syringe pump). The UV lamp was set perpendicular to the quartz tube, the length of the quartz tube exposed to the UV irradiation was estimated to be 15 cm, the longitudinal axis of the quartz tube was about 8 mm above the focal point of the UV lamp, and the residence time of the feed stream in the irradiation zone was 16 s and UV irradiation energy calculated as 1.4 MJ/kg SAP. The viscosity of the product stream was measured with a cup and bob fixture in steady mode, and at 4 s-1 it was measured as 155 mPa·s

### Preparation of PAA A10-Containing Base Polymer BP A10 of Example A10

A 2,000 ml resin kettle (equipped with a four-necked glass cover closed with septa, suited for the introduction of a thermometer, syringe needles) was placed into an ice bath filled with about 1 liter of water, 100 g of sodium chloride and about 200 g of ice such that the mixture covers about half the height of the resin kettle. The resin kettle was charged with about 1043.1 g of solution comprising aqueous PAA-A10 obtained as described above, of about 2.68% w concentration wherein the weight average molecular weight Mw determined by Gel Permeation Chromatography was 1,080 kDa (test method as described herein above). A magnetic stirrer, capable of mixing the whole content (when liquid), was added to the resin kettle and stirring was started.

The full amount of 432.1 g of glacial AA (=acrylic acid) was added to the PAA solution in the resin kettle while stirring was continued.

About 20.0 g of deionized water was taken to dissolve 0.483 g of “KPS” (=potassium peroxydisulfate, from Sigma Aldrich) e.g. in a glass vial of 40 mL volume. The vessel with the “KPS” solution was closed with a plastic snap-on cap and set aside.

About 10.0 g of deionized water was taken to dissolve 0.011 g of “ASC” (=Ascorbic Acid, from Sigma Aldrich) e.g. in a glass vial of 40 mL volume. The vessel with the “ASC” solution was closed with a plastic snap-on cap and set aside.

About 30 g of deionized water was taken to dissolve 3.22 g of “PEG700-DA” (=polyethylene glycol diacrylate of Mn˜700 Da from Sigma Aldrich) e.g. in a 50 mL glass beaker. The beaker with the “PEG700-DA” solution was covered e.g. with parafilm and set aside.

The remaining amount of water up to a final weight of 174.0 g was added to the resin kettle and stirring was continued as a homogeneous solution was obtained within 1-5 minutes.

A thermometer was introduced and in total 347.2 g of 50% w NaOH (sodium hydroxide) solution (for analysis, from Merck KGaA) were added subsequently in portions such that the temperature was below 30° C.

The “PEG700-DA” solution was added to the mixture of PAA, AA and NaOH solution at a temperature below 30° C. while stirring was continued.

Then, the resin kettle was closed, the ice bath underneath removed, and a pressure relief was provided e.g. by puncturing two syringe needles through the septa. The solution was then purged vigorously with argon via an 80 cm injection needle at about 0.4 bar while stirring at about 400 rpm. The argon stream was placed close to the stirrer for efficient and fast removal of dissolved oxygen.

After about 1 hour of Argon purging and stirring, about 0.020 g (about 1-2 droplets) of 1% w aqueous solution of hydrogen peroxide H2O2 (Sigma-Aldrich) was added via 1 mL plastic pipette to the “KPS” solution, and the latter was then added to the reaction mixture via plastic funnel inserted temporarily in one of the resin kettle cover necks while stirring and Argon purging was continued. Thereafter the “ASC” solution was added to the reaction mixture at a temperature of about 20° C. via plastic funnel inserted temporarily in one of the resin kettle cover necks while stirring and Argon purging was continued.

After the initiator solutions “KPS” and “ASC” were mixed with the reaction mixture, stirring and Argon purging was continued but the Argon needle was pulled a few cm above the liquid. Typically, within 4 min of “ASC” solution addition, the solution characteristically starts to become turbid or a sudden increase in viscosity was observed, typically at temperatures about room temperature. A “gel point” was observed and recorded when the stirbar was not able to rotate freely at the bottom of the resin kettle and the stirring was therefore stopped. Purging with argon was continued at a reduced flow rate (0.2 bar).

The temperature was monitored; typically, it rises from about 20° C. to about 80° C. within 60 minutes. Once the temperature starts to drop from a maximal value, the resin kettle was transferred into a circulation oven (e.g. Binder FED 720 from Binder GmbH) and kept at about 60° C. for about 18 hours.

After this time, the oven was switched off and the resin kettle was allowed to cool down for about 2 hours while remaining in the oven. After that, the gel was removed and broken manually or cut with scissors into smaller pieces. The gel was grinded with a grinder (X70G from Scharfen Slicing Machines GmbH with Unger R70 plate system: 3 pre-cutter kidney plates with straight holes at 17 mm diameter), put onto perforated stainless steel dishes (hole diameter 4.8 mm, 50 cm×50 cm, 0.55 mm caliper, 50% open area, from RS; max. height of gel before drying: about 3 cm) and transferred into a circulation oven (Binder FED 720 from Binder GmbH) at about 120° C. for about 20 hours.

The residual moisture content of the dried gel was about 3% by weight (see UPM test method for description of how to determine moisture content).

The dried gel was then ground using a centrifuge mill (Retsch ZM 200 from Retsch GmbH with vibratory feeder DR 100 (setting 50-60), interchangeable sieve with 1.5 mm opening settings, rotary speed 8000 rpm). The milled polymer was then sieved via a sieving machine (AS 400 control from Retsch with sieves DIN/ISO 3310-1 at about 250 rpm for about for 5-10 min) to the following particle size cuts with the following yields:

| | fines | Collected fraction | crude |
|---|---|---|---|
| sieved fraction | <150 μm | 150-710 μm | >710 μm |
| yield | | ~350 g | |

The fractions “fines” and “crude” have been discarded and not used further.

Procedure to obtain the PAA used in Example A11 (=PAA A11) from degradation of pre-existing SAP material: Liquid Whistle (LW) mediated mechanical energy degradation)

The pre-existing SAP material (in the form of pre-existing SAP particles) used for degradation is commercially available in Pampers Baby Dry as marketed in Germany in 2020.

The pre-existing SAP material was mixed with RO (=reverse osmosis) water in an agitation tank system similar to EnSight Solutions Likwifier LORSS series, equipped with approximately 20 gallon working capacity tank, top mounted scrap surface agitator, bottom 6 hole/3 wing rotor-stator high shear impeller to produce a feed stream (in the form of a gel) with 2.5 wt % SAP and 97.5 wt % RO water. The gel had a viscosity of 841 Pa·s. The feed stream was fed into the Liquid Whistle apparatus (LW; Model-A Sonolator; Sonic Corp., Stratford, CT); ellipsoidal orifice dimensions: width was 2×0.0375 in.=1.9 mm, height was 2×0.012 in.=0.6 mm (hydraulic diameter was calculated as 1.7 mm), land length was 1 mm, and volume V=π×(width)×(height)×(land length)/4=0.9 mm³) (the ellipsoidal orifice had a cross-sectional surface area of about 1.3 mm2) with flowrate of about 8 L/min and pressure of about 4,500 psi (310 bar), and the product stream was recirculated back into the agitation tank system. The tank volume was passed through the LW apparatus about 8 times, representing a total residence time of about 40 ms in the LW chamber region (about 5 ms per pass). The energy density achieved from the mixing device was about 62 MJ/m3 (about 2.48 MJ/kg SAP).

The actual final solid content of the product was determined to be 2.73% wt via placing 3.00 g thereof in a pre-weighed glass vial of 40 mL volume and placing said vial without cap inside a vacuum oven.

### Preparation of PAA A11-Containing Base Polymer BP A11 of Example A11

A 2,000 ml resin kettle (equipped with a four-necked glass cover closed with septa, suited for the introduction of a thermometer, syringe needles) was placed into an ice bath filled with about 1 liter of water, 100 g of sodium chloride and about 200 g of ice such that the mixture covers about half the height of the resin kettle. The resin kettle was charged with about 1024.0 g of solution comprising aqueous PAA A11 obtained as described above, of about 2.73% w concentration wherein the weight average molecular weight Mw determined by Gel Permeation Chromatography was 418 kDa (test method as described herein above). A magnetic stirrer, capable of mixing the whole content (when liquid), was added to the resin kettle and stirring was started.

The full amount of 432.1 g of glacial AA (=acrylic acid) was added to the PAA solution in the resin kettle while stirring was continued.

About 20.0 g of deionized water was taken to dissolve 0.484 g of “KPS” (=potassium peroxydisulfate, from Sigma Aldrich) e.g. in a glass vial of 40 mL volume. The vessel with the “KPS” solution was closed with a plastic snap-on cap and set aside.

About 10.0 g of deionized water was taken to dissolve 0.012 g of “ASC” (=Ascorbic Acid, from Sigma Aldrich) e.g. in a glass vial of 40 mL volume. The vessel with the “ASC” solution was closed with a plastic snap-on cap and set aside.

About 30 g of deionized water was taken to dissolve 3.14 g of “PEG700-DA” (=polyethylene glycol diacrylate of Mn˜700 Da from Sigma Aldrich) e.g. in a 50 mL glass beaker. The beaker with the “PEG700-DA” solution was covered e.g. with parafilm and set aside.

The remaining amount of water up to a final weight of 193.0 g was added to the resin kettle and stirring was continued as a homogeneous solution was obtained within 1-5 minutes.

A thermometer was introduced and in total 347.4 g of 50% w NaOH (sodium hydroxide) solution (for analysis, from Merck KGaA) were added subsequently in portions such that the temperature was below 30° C.

The “PEG700-DA” solution was added to the mixture of PAA, AA and NaOH solution at a temperature below 30° C. while stirring was continued.

Then, the resin kettle was closed, the ice bath underneath removed, and a pressure relief was provided e.g. by puncturing two syringe needles through the septa. The solution was then purged vigorously with argon via an 80 cm injection needle at about 0.4 bar while stirring at about 400 rpm. The argon stream was placed close to the stirrer for efficient and fast removal of dissolved oxygen.

After about 1 hour of Argon purging and stirring, about 0.025 g (about 1-2 droplets) of 1% w aqueous solution of hydrogen peroxide H2O2 (Sigma-Aldrich) was added via 1 mL plastic pipette to the “KPS” solution, and the latter was then added to the reaction mixture via plastic funnel inserted temporarily in one of the resin kettle cover necks while stirring and Argon purging was continued. Thereafter the “ASC” solution was added to the reaction mixture at a temperature of about 20° C. via plastic funnel inserted temporarily in one of the resin kettle cover necks while stirring and Argon purging was continued.

After the initiator solutions “KPS” and “ASC” were mixed with the reaction mixture, stirring and Argon purging was continued but the Argon needle was pulled a few cm above the liquid. Typically, within 3 min of “ASC” solution addition, the solution characteristically starts to become turbid or a sudden increase in viscosity was observed, typically at temperatures about room temperature. A “gel point” was observed and recorded when the stirbar was not able to rotate freely at the bottom of the resin kettle and the stirring was therefore stopped. Purging with argon was continued at a reduced flow rate (0.2 bar).

The temperature was monitored; typically, it rises from about 20° C. to about 80° C. within 60 minutes. Once the temperature starts to drop from a maximal value, the resin kettle was transferred into a circulation oven (e.g. Binder FED 720 from Binder GmbH) and kept at about 60° C. for about 18 hours.

After this time, the oven was switched off and the resin kettle was allowed to cool down for about 2 hours while remaining in the oven. After that, the gel was removed and broken manually or cut with scissors into smaller pieces. The gel was grinded with a grinder (X70G from Scharfen Slicing Machines GmbH with Unger R70 plate system: 3 pre-cutter kidney plates with straight holes at 17 mm diameter), put onto perforated stainless steel dishes (hole diameter 4.8 mm, 50 cm 50 cm, 0.55 mm caliper, 50% open area, from RS; max. height of gel before drying: about 3 cm) and transferred into a circulation oven (Binder FED 720 from Binder GmbH) at about 120° C. for about 20 hours.

The residual moisture content of the dried gel was about 3% by weight (see UPM test method for description of how to determine moisture content).

The dried gel was then ground using a centrifuge mill (Retsch ZM 200 from Retsch GmbH with vibratory feeder DR 100 (setting 50-60), interchangeable sieve with 1.5 mm opening settings, rotary speed 8000 rpm). The milled polymer was then sieved via a sieving machine (AS 400 control from Retsch with sieves DIN/ISO 3310-1 at about 250 rpm for about for 5-10 min) to the following particle size cuts with the following yields:

| | fines | Collected fraction | crude |
|---|---|---|---|
| sieved fraction | <150 μm | 150-710 μm | >710 μm |
| yield | | ~350 g | |

The fractions “fines” and “crude” have been discarded and not used further.

Surface crosslinking treatment (hereinafter referred to as “SXL”) of base polymer particles BP A1 to BP A11 and BP C1/7 to BP C6 in order to obtain examples A1 to A11 and comparative examples C1 to C7

### Equipment List

Glassware, one way pipette, spatula, spoon to prepare solution and weigh in absorbent materials
Glass Beaker: 250 ml opening ø70 mm
Balance: Sartorius or equivalent; accuracy 0.01 g
Analytical balance: Mettler or equivalent; accuracy 0.0001 g
Electrical stand stirrer: IKA Eurostar power control visc (Range 50-2000 rpm) or equivalent
With Stirrer: PTFE Propeller stirrer 4-bladed _ø 50 mm
Pipette: Eppendorf Multi stream or equivalent
Aluminum foil for covering
Circulation oven: Binder FD 240 or equivalent
Equipment to determine Moisture: Halogen Moisture Balance Mettler or equivalent
Sieve machine: Retch AS 200 control “g” or equivalent
With Sieves: stainless steel: DIN/ISO 3310-1 ø10 mm

### Preparation of Solutions

Aluminum Lactate Solution

Prepare 1 kg 15 wt % Aluminum lactate stock solution in deionized water (MilliporeQ of conductivity <1.6 μS/cm) by adding 850 g of deionized water to 150 g of Aluminum lactate.
Surface crosslinking solutions (SXL solutions) (see table 2):

The used Denacol concentrations were prepared according to Table 2, each in snap cap jars of volume about 50 ml.

To prepare the solutions, the Denacol bottle or container (ca. 1 L) was taken out of the fridge and let to stay out to thermally equilibrate for ca. 30 min before preparing the solutions.

Solutions were prepared as follows:

Different respective concentrations, for the given examples, of Denacol EX-810, DN-810 ex Nagase Co. Ltd.) were prepared by adding the amount shown in Table 2 to the snap cap plastic jar which was then filled to 20 g with 1,2-Propanediol (Merck KGaA).

TABLE 2

| Surface | Examples/ | Concentration | |
|---|---|---|---|
| crosslinking agent | comparative examples | (in wt. %) | Preparation of solution |
| Denacol EX-810 | A1-A6, A9-A11, C2, | 8% | 1.6 g DN-810 filled in with |
| | C6, C7 | | 1,2 Propanediol to 20 g |
| Denacol EX-810 | A7 | 6% | 1.2 g DN-810 filled in with |
| | | | 1,2 Propanediol to 20 g |
| Denacol EX-810 | C5 | 5% | 1.0 g DN-810 filled in with |
| | | | 1,2 Propanediol to 20 g |
| Denacol EX-810 | C1 | 4% | 0.8 g DN-810 filled in with |
| | | | 1,2 Propanediol to 20 g |
| Denacol EX-810 | A8, C3-C4 | 7% | 1.4 g DN-810 filled in with |
| | | | 1,2 Propanediol to 20 g |

### Execution of SXL Procedure

Each of the respective dry base polymer particles BP A1 to BP A11 and BP C1 to BP C7 was weighed to be 20-30 g and recorded to ±0.1 g and placed in a separate 250 ml glass beaker so that the filling height is ≤25% of the overall height. Exact amounts are given in Table 4.

The base polymer particles were mixed at 600+/−50 rpm with a PTFE stirrer into the beaker. The stirrer was just touching the bottom of the beaker. The base polymer particles needed to be stirred until good fluidization of the bed is achieved.

The requested amounts of solutions were added with an Eppendorf pipette, step by step like described below and the actual quantities are given in Table 4. (Speed setting of Eppendorf pipette: Middle speed)

### Step 1

The amount of Aluminum Lactate Solution was added into the center of stirring agitation. Afterwards, the stirring speed was raised to 2000 +/−50 rpm. Stirred for approximately 15 seconds and continued with Step 2. If necessary, covered beaker with e.g. aluminum foil to avoid jumping out of material.

### Step 2

The amount of SXL solution was added into the center of stirring agitation. Stirred for approximately 15 seconds and continued with Step 3.

### Step 3

Amount of deionized water (3 wt % based on sample weight) was added into the center of stirring agitation. Stirred for approximately 15 seconds. After stopping stirrer transferred the material into a heat resistant wide-mouth glass vial (e.g. crystallizing dish) and distributed it evenly. Took loose material only and left strong stacked material on wall in beaker. Removed loose material by slight tapping outside on wall of beaker or by use of spatula. Avoided scratching out. Covered the wide mouth glass vial with aluminum foil and stored it into a fume hood at room temperature for approximately 16 h to 18 h (overnight is recommended) and afterwards heated the material in the oven at requested temperature and time (e.g. Surface crosslinking Denacol heat up period of 20 min from room temperature to 120° C. in addition to the 3 h heating time).

After 2 h 20 min heating time, the aluminum foil was half-way open and stayed like this for the remaining 1 h of heating to drive moisture lower than 1% w.

After heating time, removed container from the oven and placed the material into a fume hood to cool down to room temperature, for approximately 15 min.

The final polymers were tested for moisture, the results are shown in Table 3.

TABLE 3

| Example | Moisture, wt % |
|---|---|
| A1 | 0.4 |
| A2 | 0.4 |
| A3 | 0.4 |
| A4 | 0.4 |
| A5 | 0.4 |
| A6 | 0.4 |
| A7 | 0.4 |
| A8 | 0.5 |
| A9 | 0.4 |
| A10 | 0.4 |
| A11 | 0.5 |
| C1 | 0.2 |
| C2 | 0.8 |
| C3 | 0.7 |
| C4 | 0.6 |
| C5 | 0.6 |
| C6 | 0.6 |
| C7 | 0.5 |

TABLE 4

| | Amount of | | Overall | Overall | |
|---|---|---|---|---|---|
| | Base | | Denacol | Aluminium | Added |
| | Polymer | Denacol EX- | Ex810 add- | lactate add- | deionized |
| Examples/ | Particles | 810 | on vs. | on vs | water |
| comparative | treated | concentration | polymer (in | polymer (in | (in wt % vs |
| examples | (in g) | (in wt %) | wt %) | wt %) | polymer) |
| A1-A6, A9-A11, C7 | 30.0 | 8% | 0.080 | 0.5 | 12.5 |
| C1 | 30.0 | 4% | 0.040 | 0.5 | 12.5 |
| C2 | 20.0 | 8% | 0.144 | 0.9 | 12.5 |
| C5 | 30.0 | 5% | 0.050 | 0.5 | 12.5 |
| C6 | 30.0 | 8% | 0.080 | 0.9 | 12.5 |
| A7 | 30.0 | 6% | 0.060 | 0.5 | 12.5 |
| A8, C3-C4 | 30.0 | 7% | 0.070 | 0.5 | 12.5 |

The quantities of Denacol EX-810 and Aluminum lactate add-on were selected such that the resulting examples and comparative examples exhibited SFC above 1 unit and CRC preferably above 18 g/g. (see Table 5)

TABLE 5
Performance of Examples A1 to A11 and Comparative Examples C1 to C7

| | | | | Ratio | | | |
|---|---|---|---|---|---|---|---|
| | | | | (Extract.1) | | | |
| | | | | minus s-PAA | | | |
| | | | s-PAA | add-on level | | | |
| | CRC1), | Extractables1), | add-on | [% w]) to | CRC2), | EFFC2), | UPM2), |
| EXAMPLE | g/g | % w | level, % wt | CRC1) [g/g] | g/g | g/g | g/g |
| A1 | 44.1 | 8.2 | 5 | 0.07 | 32 | 28.5 | 21 |
| A2 | 43.1 | 8.4 | 5 | 0.08 | 28.8 | 26.2 | 54 |
| A3 | 37.6 | 7.9 | 10 | −0.06 | 27 | 25.0 | 87 |
| A4 | 43.7 | 9.2 | 10 | −0.02 | 29.4 | 26.7 | 39 |
| A5 | 38.1 | 11.0 | 30 | −0.50 | 26.3 | 23.5 | 52 |
| A6 | 40.9 | 8.7 | 15 | −0.15 | 27.7 | 25.4 | 62 |
| A7 | 35.9 | 9.1 | 20 | −0.30 | 27.4 | 24.5 | 35 |
| A8 | 35.6 | 14.9 | 20 | −0.14 | 24.7 | 22.6 | 111 |
| A9 | 22 | 10.6 | 66.7 | −2.55 | 18 | 16.5 | 126 |
| A10 | 42.9 | 9.7 | 5 | 0.11 | 30.9 | 27.4 | 27 |
| A11 | 44.1 | 9.9 | 5 | 0.11 | 30.2 | 27.1 | 36 |
| C1 | 46.3 | 11.1 | 0 | 0.24 | 34.7 | 28.3 | 4 |
| C2 | 35.3 | 7.4 | 0 | 0.21 | 26.3 | 23.9 | 54 |
| C3 | 44.8 | 13.9 | 5 | 0.20 | 31.9 | 26.9 | 20 |
| C4 | 51.6 | 24.6 | 10 | 0.28 | 36.6 | 26.1 | 2 |
| C5 | 45.8 | 13.6 | 5 | 0.19 | 33 | 27.7 | 16 |
| C6 | 47.1 | 22.0 | 10 | 0.26 | 32.6 | 25.1 | 9 |
| C7 | 46.3 | 11.1 | 0 | 0.24 | 29.9 | 26.5 | 31 |

1)Value for base polymer particle
2)Value for SAP particle after surface cross-linking

As is shown by the data in Table 5, the SAP particles of Examples A1 to A11 all exhibit good properties in terms of capacity (CRC), EFFC and permeability (UPM). The amount of extractables for comparable add-on level of the s-PAA polymers is significantly lower, as can be seen e.g. by comparing the amount of extractables of examples A1, A2, A10 and A11 with comparative examples C3 and C5, all having an add-on level of s-PAA polymers of 5 weight-%.

This is also reflected by the ratio of (extractables minus s-PAA polymer add-on level) to CRC of the base polymer. This ratio reflects the impact of the add-on level of the s-PAA polymers on the overall amount of extractables-and put in relation to the capacity (as an increase of capacity generally leads to an increase of amount of extractables in SAP particles). Basically, the amount of extractables of comparative examples C3 and C5 is roughly 5 weight-% higher than the amount of extractables of examples A1 and A2, indicating that the s-PAA polymers of the comparative examples have leaked out of the SAP particles to a very high extent. Compared thereto, the s-PAA polymers of the inventive examples did not significantly leak out of the SAP particles, indicating that they are covalently bound into the network due to their carbon-to-carbon double bonds. By applying s-PAA polymers having carbon-to-carbon double bonds, even s-PAA polymers of relatively low average weight molecular weight do not significantly contribute to the amount of extractables, as can especially be seen in example A1, which has an average molecular weight as low as 134 kDa. Typically, molecules with low average weight molecular weight have a higher likelihood of leaking (thus contributing to the amount of extractables) as they can escape more readily out of a swollen polymer network. However, being able to get polymerized into the polymer network of the SAP particles due to their carbon-to-carbon double bonds, even such relatively small s-PAA polymers can be readily used in the making of SAP particles.

Moreover, as s-PAA polymers having carbon-to-carbon double bonds can act to crosslink polymer chains during polymerization, therefore enabling the reduction or even elimination of additional cross-linkers that are commonly applied in the making of SAP material. This is reflected by the results of examples A5 and A7 (reduced amount of-additional-crosslinker vs. 0.075 mol. ratio) and of examples A8 and A9 (no-additional-crosslinker), which all exhibit good properties.

The examples having the lowest molar percent of carbon-to-carbon double bonds among the inventive examples, namely A10 and A11, have a relatively higher amount of extractables, as well as higher ratio of (extractables minus s-PAA polymer add-on level) to CRC of the base polymer compared to the other inventive examples. However, these examples still have a considerably better ratio of ratio of (extractables minus s-PAA polymer add-on level) to CRC of the base polymer than the comparative examples.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.” Further, every numerical range given throughout this specification includes every narrower numerical range that falls within such broader numerical range.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of making superabsorbent polymer material, the method comprising the steps of:
a) providing an aqueous solution of polymerizable acrylic acid monomers and/or polymerizable acrylic acid oligomers, optionally neutralizing at least some of the polymerizable acrylic acid monomers and/or polymerizable acrylic acid oligomers;
b) optionally providing one or more ethylenically unsatured co-monomers, optionally neutralizing at least some of the ethylenically unsatured co-monomer of step b);
c) optionally providing one or more crosslinker(s);
d) providing one or more initiator(s);
e) providing soluble polyacrylic acid polymers, wherein the soluble polyacrylic acid polymers have a molar percent of carbon-to-carbon double bonds of at least about 0.02;
f) mixing the aqueous solution of monomers, oligomers, co-monomers, crosslinkers and initiators and soluble polyacrylic acid polymers provided in steps a) to e); and
g) polymerizing the mixture obtained in step f) to obtain a superabsorbent polymer.

2. The method of claim 1, wherein the soluble polyacrylic acid polymers provided in step e) are provided at a weight-percent of at least about 3 weight-%, preferably at least about 5 weight-% based on the total weight of the soluble polyacrylic acid polymers provided in step e) and the monomers, oligomers, co-monomers, crosslinkers and initiators provided in steps a) to d).

3. The method of claim 1, wherein the soluble polyacrylic acid polymers provided in step e) are provided at a weight-percent of up to about 60.0 weight-%, based on the total weight of the soluble polyacrylic acid polymers provided in step e) and the polymerizable acrylic acid monomers, polymerizable acrylic acid oligomers, ethylenically unsatured co-monomers, crosslinkers, and initiators provided in steps a) to d).

4. The method of claim 1, wherein the method further comprises a step h) of drying the superabsorbent polymer material.

5. The method of claim 1, further comprising a step i) of comminuting the superabsorbent polymer material to obtain superabsorbent polymer particles.

6. The method of claim 5, further comprising the step of surface cross-linking the superabsorbent polymer particles.

7. The method of claim 1, wherein the soluble polyacrylic acid polymers are obtained from pre-existing recycled post-consumer superabsorbent polymer material, and/or obtained from pre-existing recycled post-industrial superabsorbent polymer material.

8. The method of claim 7, wherein the soluble polyacrylic acid polymers are obtained from pre-existing recycled post-consumer superabsorbent polymer material, and/or obtained from pre-existing recycled post-industrial superabsorbent polymer material by chemical degradation, UV degradation, mechanical degradation, or combinations thereof.

9. The method of claim 7, wherein the method further comprises the step of al) obtaining the soluble polyacrylic acid polymers from pre-existing recycled post-consumer superabsorbent polymer material or from pre-existing recycled post-industrial superabsorbent polymer material by chemical degradation of the pre-existing recycled post-consumer superabsorbent polymer material, and wherein step a1) is carried out prior to step b).

10. The method of claim 8, wherein the chemical degradation is carried out with an oxidative water-soluble salt comprising at least one cation and at least one anion.

11. The method of claim 9, wherein the at least one anion is selected from the group consisting of: peroxydisulfate, peroxymonosulfate, peroxydicarbonate, peroxydiphosphate, peroxydiborate and mixtures and combinations thereof.

12. The method of claim 8, wherein the chemical degradation is mediated by redox couples, wherein the redox couples are selected from the group consisting of sodium peroxodisulfate/ascorbic acid; hydrogen peroxide/ascorbic acid; potassium peroxodisulfate/sodium bisulfite; sodium peroxodisulfate/sodium bisulfite; hydrogen peroxide/sodium bisulfite; potassium peroxodisulfate/ascorbic acid and combinations thereof.

13. The method of claim 1, wherein the soluble polyacrylic acid polymers have a weight average molecular weight Mw of from about 500 kDa to about 3 MDa.

14. The method of claim 1, wherein the superabsorbent polymer material obtained by the method has an amount of extractables of less than about 15.0 weight-% based on the total weight of the superabsorbent polymer material, and the ratio of (amount of the difference between extractables (weight-%) and weight-% of soluble polyacrylic acid polymers provided in step e)) to capacity (g/g) of less than 0.15.

15. The method of claim 1, wherein the superabsorbent polymer material obtained by the method has a capacity measured as Centrifuge Retention Capacity (CRC) in accordance the test method set out herein of at least about 20 g/g.

16. The method of claim 1, wherein the polymerizable acrylic acid monomers and/or polymerizable acrylic acid oligomers provided in method step a) are neutralized at a degree of neutralization from 40 to 95 mol %.

17. The method of claim 1, wherein the soluble polyacrylic acid polymers provided in step e) may have a weight average molecular weight Mw of at least 50 kDa.

18. The method of claim 1, wherein the soluble polyacrylic acid polymers are provided in step e) at a weight-percent of at least 3 weight-% based on the total weight of the soluble polyacrylic acid polymers provided in step e) and the polymerizable acrylic acid monomers, polymerizable acrylic acid oligomers, ethylenically unsatured co-monomers, crosslinkers, and initiators provided in steps a) to d).

19. The method of claim 1, wherein the superabsorbent polymer has an amount of extractables of less than 15.0 weight-% based on the total weight of the superabsorbent polymer.

20. The method of claim 1, comprising providing one or more ethylenically unsatured co-monomers, wherein the one or more ethylenically unsatured co-monomers are provided at less than 3-weight-% based on the total weight of the polymerizable acrylic acid monomers and/or polymerizable acrylic acid oligomers.
